(19) United States
(12) Patent Application Publication
(10) Pub. No.: US 2024/0274256 A1
(43) Pub. Date: Aug. 15, 2024

(54) MACHINE LEARNING SYSTEM AND METHOD TO DETERMINE STEP UP AND STEP DOWN OF TREATMENTS

(71) Applicants: RECIPROCAL LABS CORPORATION, San Diego, CA (US); RESMED INC., San Diego, CA (US)

(72) Inventors: Sibel Adelina SAYINER, San Diego, CA (US); Nicholas John HIRONS, London (GB); Meredith Ann BARRETT, San Diego, CA (US); Se'Shanel CHILDS, San Diego, CA (US); Yeqing WANG, San Diego, CA (US); Ian J. THOMAS, San Diego, CA (US); Leanne KAYE, San Diego, CA (US); Melissa WILLIAMS, San Diego, CA (US); Noah MATSUYOSHI, San Diego, CA (US); Susa V-MONACELLI, San Diego, CA (US); Theodore BURNS, San Diego, CA (US); Vy VUONG, San Diego, CA (US)

(21) Appl. No.: 18/568,209

(22) PCT Filed: Jun. 7, 2022
(86) PCT No.: PCT/US2022/032547

§ 371 Date: Dec. 7, 2023

Related U.S. Application Data
(60) Provisional application No. 63/197,858, filed on Jun. 7, 2021.

(51) Int. Cl. G16H 20/13; G16H 10/60; G16H 40/67

(52) U.S. Cl. CPC G16H 20/13; G16H 10/60; G16H 40/67

(57) ABSTRACT

A system and method to determine a recommendation to change a treatment regimen of a respiratory ailment. The treatment regimen includes multiple steps. Use data of a respiration medicament device to deliver controller or rescue respiration medicament to a patient is collected via a communication interface. The use data is transmitted to a storage device. The use data in the storage device is made accessible to a data analysis module. A patient value associated with the treatment regimen is determined based on the collected use data and patient context data. A comparison of the patient value is made to a threshold level. A recommendation to change the step of the treatment is made based on the comparison to the threshold level. A notification of recommendation of the change of the step of the treatment regimen is provided.

### PRIORITY CLAIM

The present disclosure claim priority to and benefit of U.S. Provisional Patent Application No. 63/197,858 filed on Jun. 7, 2021. The contents of that application are hereby incorporated by reference in their entirety.

### TECHNICAL FIELD

The present disclosure relates generally to methods of improving treatment for patients with respiratory ailments, and more specifically to determining changing appropriate treatment levels based on collecting data from multiple sources.

### BACKGROUND

Respiratory ailments such as asthma remain a significant and costly public health problem. For example, in the United States, more than 22 million people have asthma. Worldwide, the World Health Organization estimates the population with asthma may be 235 million, and predicts that it will rise in the future. Similarly, recent studies by the Centers for Disease Control and Prevention have listed COPD as the third leading cause of death in the United States while estimating close to 15 million people may have COPD induced impaired lung function (Wheaton, A. G., Cunningham, T. J., Ford, E. S., Croft, J. B., Employment and activity limitations among adults with chronic obstructive pulmonary disease—United States, 2013. MMWR Morb. Mortal Wkly. Rep., 2015, 64(11), 290-295).

Despite the development of new medications, rates of hospitalizations and emergency room visits have not declined. Each year in the United States asthma causes approximately 2 million emergency department visits, 500,000 hospitalizations, and 5,000 deaths. In addition, asthma is responsible for an estimated 5.2 million missed days of school, and 8.7 million days of work (Nurmagambetov, T., Kuwahara, R., Garbe, P., The Economic Burden of Asthma in the United States, 2008-2013, Ann. Am. Thorac. Soc., 2018, 15(3), 348-356). Total annual costs to US health insurers and employers are greater than US$82 billion (ibid). COPD causes approximately 715,000 hospitalizations, and 134,000 deaths yearly. Additionally, the cost to the nation for COPD was recently projected to be approximately US $49.9 billion, including US $29.5 billion in direct health care expenditures, US$8.0 billion in indirect morbidity costs and US $12.4 billion in indirect mortality costs.

Asthma can be effectively treated: patients who achieve good control of their asthma have minimal symptoms and avoid exacerbations, allowing them to live normal lives. Despite the preventability of asthma exacerbations with currently available treatments, only 1 in 5 asthmatics has the disease under control. Such treatments often rely on a patient to properly self-administer a medicament, such an inhaler. When a patient is exhibiting symptoms, a patient may identify that their asthma is being triggered and may administer a “reliever” medicament via a puff from an inhaler.

Revised national guidelines urge doctors to more closely monitor whether prescribed treatment for asthma is controlling everyday symptoms and improving quality of life. In addition, providers are instructed to collect broad information about a patient to guide their decision-making before making a medication adjustment and at a regular cadence after any adjustments (e.g., 2-3 months). For example, the Global Initiative for Asthma (GINA) guidelines recommends that providers assess: a confirmation of disease diagnosis, exacerbation history, lung function, comorbidities and any relevant treatments, tobacco use or exposure, environmental risk factors, allergies, inhaler technique, medication history, medication side effects, patient self-management goals and patient satisfaction.

Healthcare providers, however, are limited by time and an availability of tools to assess these wide-ranging factors. Providers are also limited in available tools to evaluate how their patients are doing on a day-to-day basis. An increasing number of physicians have begun to use periodic, written questionnaires (e.g., the Asthma Control Test (ACT)) to monitor patients and determine their level of control. These instruments require patients to accurately recall and report the frequency of symptoms, medicament device (e.g., inhaler) usage, and activity level and restriction over some period of time, usually two to four weeks. As a result, collected information relating to management of respiratory disorders has been subject to error introduced by biases (e.g., fallible recollection), different interpretations of symptoms, and behaviors (e.g., non-adherence), and extremely limited frequency of data collection such that the data is of limited utility.

In respiratory disease, patients are primarily managed through medication and are “stepped up” or “stepped down” in type of medication or dosage based on clinical indications, such as symptom levels and prior medication usage. Though these considerations for the appropriate step of the treatment are generally outlined in clinical guidelines, such as GINA, or the National Heart, Lung, and Blood Institute's National Asthma Education and Prevention Program Coordinating Committee Expert Panel Working Groups (NHLBI NAEPPCC), there is a lack of objective data collection to inform these decisions. Thus, healthcare providers may often have to make subjective decisions as to whether a treatment regimen should be continued, stepped-up or stepped-down, leading to potential bias and sub-optimal management of patients based on a paucity of information. Thus, patients with very similar clinical status may receive different recommendations for treatment management even with the same healthcare provider. This has led to more than 60% of patients with current asthma remaining uncontrolled or being unnecessarily stepped up to higher dosage drugs, both detrimental to their health and clinical outcomes.

Currently, patient data may be collected from sensors or from electronic health records (EHR). However, neither of these methods allows for an accurate assessment of treatment, nor do they capture the breadth of information recommended by guidelines as described previously. For example, data obtained through smart sensors captures usage, but does not make it actionable. Similarly, health care record applications fail to capture information relevant for respiratory condition control status.

In asthma, prior to a patient being “stepped up” to the highest strength medications, i.e., biologics, a prior authorization (PA) form is filled out by a provider to verify the necessity of such treatment. Insurance companies require these authorizations due to the high cost of the treatments, requesting information such as a blood test indicating the appropriate level of allergic response, evidence of severe symptomatic status, and adherence to the current prescribed treatment regimen. The confirmation of adherence and a patient's symptomatic status are not captured within the EHR for a patient. These are instead often physician attested, often without objective knowledge of the information. Pharmacy fill records can show whether a patient filled a medication, but does not indicate actuation. Since relevant information is thus not available, decisions to step-up or step-down treatment are not optimal.

There is a need for a system that allows for accurate determination of patients that may benefit from a step-up or step-down in treatments for different diseases and conditions. There is also a need for a system that provides analysis of the level of treatment for comprehensive management to all step-up and step-down decisions. There is a further need for system that captures and make actionable the broad range of information needed for respiratory specific use cases in determination of a step-up or step-down in treatments.

### SUMMARY

One disclosed example is a method of determining a recommendation to change a treatment regimen of a respiratory ailment. The treatment regimen includes a plurality of steps. Use data of a respiration medicament device to deliver controller or rescue respiration medicament to the patient is collected via a communication interface. The use data is transmitted to a storage device. The use data is stored in the storage device, which is accessible to a data analysis engine. Based on the collected use data and patient context data, a patient value associated with the treatment regimen is determined by the data analysis engine. A comparison of the patient value is made to a threshold level. A recommendation to change the step of the treatment regimen is made based on the comparison to the threshold level. A notification of the recommendation of the change of the step of the treatment regimen is provided.

A further implementation of the example method is where the respiratory ailment is asthma or COPD. Another implementation is where each step of the treatment regimen includes an associated controller respiration medicament or a rescue respiration medicament. Another implementation is where the treatment regimen is defined by Global Initiative for Asthma (GINA) guidelines or the National Heart Lung Blood Institute guidelines. Another implementation is where the threshold level is one of an adherence percentage over a period of time, a number of rescue medicaments over a period of time, a score on a respiratory test, or a visit to a treatment facility. Another implementation is where the respiratory test is one of the COPD assessment test (CAT) or the asthma control test (ACT). Another implementation is where the recommendation is a step-up in treatment when the comparison is above the threshold level. Another implementation is where the recommendation is a step-down in treatment when the comparison is below the threshold level. Another implementation is where the patient context data is one of demographic data, environmental data, weather data, and social determinants of health. Another implementation is where the demographic data is collected from interfacing with an electronic health record system or self-reported by the patient. Another implementation is where the recommendation of the change is made at a predetermined interval over a period of time. Another implementation is where the example method further includes inputting the collected data to a machine learning model to output the threshold value. The machine learning model is trained from collected context data, use data, and outcomes from the treatment regimen from a population of patients. Another implementation is where the machine learning model includes a clinical decision label for the threshold output. Another implementation is where the machine learning model is based on one of a generalized linear model, a tree-based model or a neural network. Another implementation is where the example method further includes categorizing the use between preemptive or emergency use. Another implementation is where the example method further includes outputting a report of the collected use and context data. Another implementation is where the example method further includes collecting the context data through a survey displayed by an application executed by a mobile computing device operated by the patient. Another implementation is where the notification is provided electronically to one of the patient, a caregiver, or a healthcare provider. Another implementation is where the example method further includes ordering additional medication for the patient through a medication supply system based on the recommendation.

Another disclosed example is a computer program product comprising instructions which, when executed by a computer, cause the computer to carry out the above described methods. Another implementation of the example computer program product is where the computer program product is a non-transitory computer readable medium.

Another disclosed example is a system to determine changing a treatment regimen having a plurality of treatment steps. The system includes a communication interface to collect use data of a respiration medicament device to deliver controller or rescue respiration medicament to a patient. A storage device stores the collected use data. A data analysis engine is configured to input the use data and context data of patient to determine a patient value. The data analysis engine compares the patient value with a threshold value and based on the comparison, makes a recommendation to change the treatment regimen. The data analysis engine provides a notification of the recommendation.

A further implementation of the example system is where the respiratory ailment is asthma or COPD. Another implementation is where each step of the treatment regimen includes an associated controller respiration medicament or a rescue respiration medicament. Another implementation is where the treatment regimen is defined by Global Initiative for Asthma (GINA) guidelines or the National Heart Lung Blood Institute guidelines. Another implementation is where the threshold level is one of an adherence percentage over a period of time, a number of rescue medicaments over a period of time, a score on a respiratory test, or a visit to a treatment facility. Another implementation is where the respiratory test is one of the COPD assessment test (CAT) or the asthma control test (ACT). Another implementation is where the recommendation is a step-up in treatment when the comparison is above the threshold level. Another implementation is where the recommendation is a step-down in treatment when the comparison is below the threshold level. Another implementation is where the patient context data is one of demographic data, environmental data, weather data, and social determinants of health. Another implementation is where the demographic data is collected from interfacing with an electronic health record system or self-reported by the patient. Another implementation is where the recommendation of the change is made at a predetermined interval over a period of time. Another implementation is where the data analysis engine determines the threshold value from inputting the collected data to a machine learning model to output the threshold value. The machine learning model is trained from collected context data, use data, and outcomes from the treatment regimen from a population of patients. Another implementation is where the machine learning model includes a clinical decision label for the recommendation output. Another implementation is where the machine learning model is based on one of a generalized linear model, a tree-based model or a neural network. Another implementation is where the data analysis engine is further configured to categorize the use between preemptive or emergency use. Another implementation is where the data analysis engine is further configured to output a report of the collected use and context data. Another implementation is where the context data is collected through a survey displayed by an application executed by a mobile computing device operated by the patient. Another implementation is where the notification is provided electronically to one of the patient, a caregiver, or a healthcare provider. Another implementation is where the data analysis engine is further configured to order additional medication for the patient through a medication supply system based on the recommendation.

Another disclosed example is a method of training a machine learning model to determine a threshold for recommendation of one step of a multiple step treatment regimen for a respiratory ailment. Use data is collected from the use of a respiratory medication application device from each of a population of patients undergoing one of the steps of the multiple step treatment regimen. Contextual data from each of the population of patients is collected. Outcomes from the population of patients for the treatment regimen are collected. Initial recommendation thresholds for each of the steps of the multiple step treatment regimen are determined. The initial recommendation thresholds are adjusted based on the collected use data, contextual data, and outcomes until a predetermined level of confidence is reached.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 shows a respiratory ailment analytics system for monitoring accurate, real-time medicament device usage, performing analytics on that data, and providing analysis of patients for which treatments should be stepped up or stepped down;

FIG. 2 is a high-level block diagram illustrating an example of a computing device used in either as a client device, application server, and/or database server;

FIG. 3 is a diagram showing the sources of data for aggregation of a determination of step-up or step-down for medication;

FIGS. 4A-4B are a diagram showing a system that gathers data for aggregation of a determination of step-up or step-down for treatment;

FIG. 5 is a block diagram of a recommendation algorithm;

FIG. 6A is a screen image of an example patient summary interface for displaying step-up or step-down recommendations for patients associated with a healthcare provider;

FIG. 6B is a screen image of an example patient data interface;

FIG. 6C is a screen image of an example recommendations details interface;

FIG. 6D is a screen image of an example threshold notification selection interface for step-up and step-down treatments;

FIG. 7A is a series of screen images of interfaces for instructing a patient on making an injection of medication;

FIG. 7B is a series of screen images allowing a patient to record the injection of medication;

FIG. 7C is a series of screen images allowing a patient to record information for determining potential exacerbations;

FIG. 7D is a table of indicators to evaluate quality of care for determination of step-up or step-down for treatment; and

FIG. 8 is a flow diagram of an example routine for determining a recommendation in changes to a treatment regimen.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a system that collects data relating to individual-level patterns of rescue inhaler use and controller medication adherence in the process of a multi-tier treatment process. The pattern data is aggregated with other relevant contextual data provided by both the individual and outside sources such as medical records. Automated analysis of the aggregated data provides an objective recommendation for either a step-up or step-down to different tiers of the treatment regimen. The step-up or step-down of treatment increases the effectiveness of the treatment regimen for respiratory ailments including the injection of biologics.

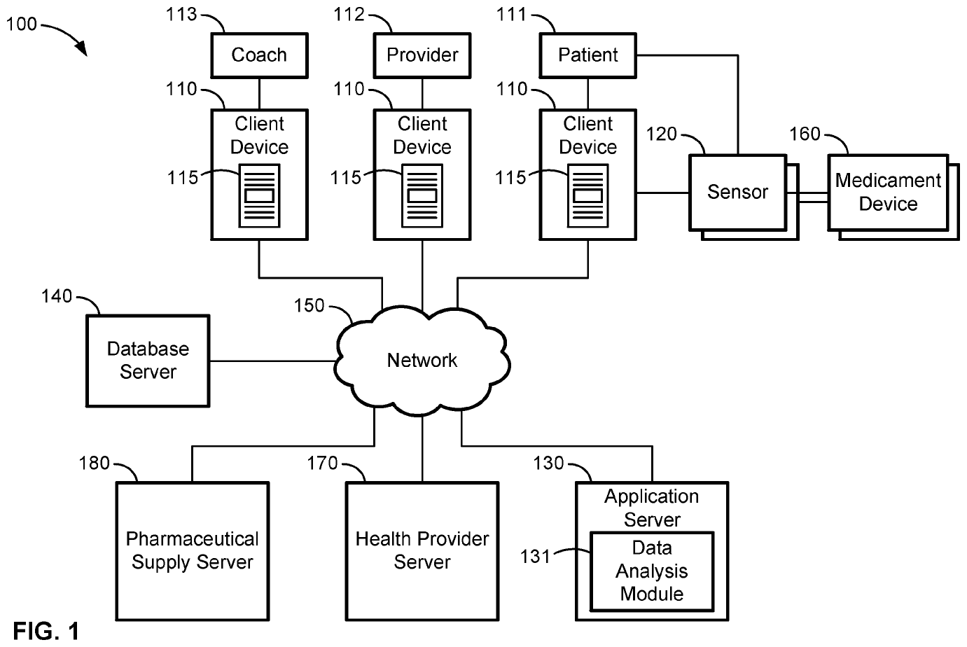

FIG. 1 shows a respiratory ailment analytics system 100 for monitoring accurate, real-time medicament device events, performing analytics on that data, and providing objective recommendations for a patient in relation to a treatment regimen based on a wide range of collected data. Such respiratory ailments may be an asthma event that may be addressed through different tiers of treatments such as medication taken orally, medicament taken through an inhaler or other devices, and injection of biologics. The example analytics system 100 allows for an objective determination to be made for stepping-up or stepping down between tiers of treatment, thus supplanting the previous subjective judgment of a health care professional.

The respiratory ailment analytics system 100 includes client computing devices 110, a medicament device sensor 120, a medicament administration device 160, an application server 130, database server 140, and a network 150. Other servers such as a health provider server 170 and a pharmaceutical supply server 180 may be coupled to the network 150. Although FIG. 1 illustrates only a single instance of most of the components of the respiratory ailment analytics system 100, in practice more than one of each component may be present, and additional or fewer components may be used.

The client devices 110, at the behest of their users, interact with the respiratory ailment analytics system 100 via the network 150. For purposes of explanation and clarity it is useful to identify at least two different types of users. A patient 111 is a user burdened with a respiratory ailment such as asthma or COPD who makes use of the respiratory ailment analytics system 100 at least in part to obtain personalized rescue event risk notifications provided by the server 130 and management notifications created by their healthcare provider 112 in this example. The patient 111 may be supported by a caregiver who is an additional or alternative user. The caregiver may have their own application 115 and client device 110 which receives the same notifications as the patient 111 (not shown in FIG. 1), or receives notifications on the patient's behalf (e.g., parents/guardians of patients 111 who may also want to receive notifications in the event that their own client devices 110 are distinct from that of their children). Such notifications can be provided in exchange for the user's permission to allow the respiratory ailment analytics system 100 to monitor the patient's 111 medicament device 160 usage. As will be explained below, in this example, medication events are detected by a sensor 120 associated with the medicament device 160 and the user's client device 110, which in turn reports to the application server 130, which in turn can initiate a process to generate risk notifications which are provided to the user through the client device 110. Other medication events for other types of medication may be determined through other types of data. In this example, the patients 111 represent a patient population, for which individual data is taken for each patient in the group.

Another type of user is a healthcare provider 112 who, again with the express permission of the patient 111, also receives notifications regarding a patient's respiratory ailment management such as asthma management, as well as aggregated asthma community rescue event data and derived statistics regarding asthma events and other associated data. Other types of users may be contemplated. Another type of user is a coach 113 that motivates the patient 111 to comply with a respiratory treatment regimen.

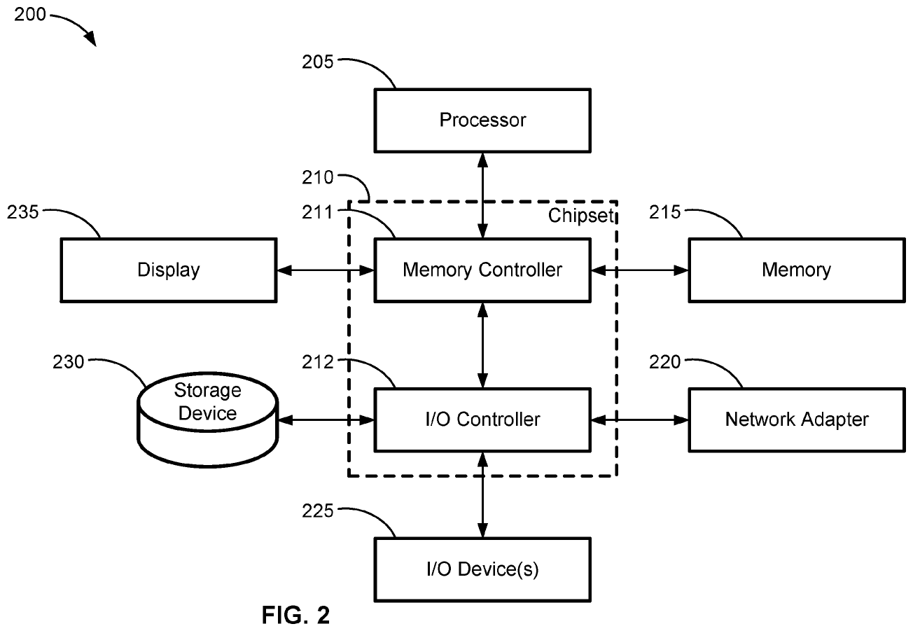

The client device 110 is a computer system that may be a portable computing device such as a smart phone, tablet, or laptop. The client device 110 could also be a television (e.g., a smart, connected television) or a speaker system (e.g., a smart, connected speaker). An example physical implementation is described more completely below with respect to FIG. 2. The client device 110 is configured to wirelessly communicate with the respiratory ailment analytics system 100 via the network 150. With network 150 access, the client device 110 transmits the time of medication usage events to system 100, as well as information describing the event as received from the associated medicament device sensor 120 (referred to throughout as “sensor 120”). The user's geographical location may also be transmitted to the respiratory ailment analytics system 100.

Regarding user location and event times, the client device 110 may determine the geographical location and time of a rescue event through use of information about the cellular or wireless network 150 to which it is connected. For example, the current geographical location of the client device 110 may be determined by directly querying the software stack providing the network 150 connection. Alternatively, the geographical location information may be obtained by pinging an external web service (not shown in FIG. 1) made accessible via network 150. The time of an event can be provided by the sensor 120 as part of the event data or added to event data by querying an appropriate software routine available as part of the client device's native operating system.

In addition to communicating with the application server 130, client devices 110 connected wirelessly to the respiratory ailment analytics system 100 may also exchange information with other connected client devices 110. For example, through a client software application 115, a healthcare provider 112 or caregiver may receive a risk exacerbation notification describing a recent rescue event about a patient 111, then in response send a recommendation to the patient 111 for post-asthma rescue event treatment. Similarly, through application 115 patients 111 may communicate with their healthcare providers 112 and other patients 111 or caregivers.

Application 115 provides a user interface (herein referred to as a “dashboard”) that is displayed on a screen of the client device 110 and allows a user to input commands to control the operation of the application 115. The dashboard is the mechanism by which healthcare providers 112, caregivers and patients 111 access the respiratory ailment analytics system 100. For example, the dashboard allows patients 111, caregivers and providers 112 to interact with each other, receive asthma rescue event risk notifications, exchange messages about treatment, provide and receive additional event and non-event data, and so on. Application 115 may be coded as a web page, series of web pages, or content otherwise coded to render within an internet browser. Application 115 may also be coded as a proprietary application configured to operate on the native operating system of the client device 110.

In addition to providing the dashboard, application 115 may also perform some data processing on asthma rescue event data locally using the resources of client device 110 before sending the processed data through the network 150. Event data sent through the network 150 is received by the application server 130 where it is analyzed and processed for storage and retrieval in conjunction with database server 140. The application server 130 may direct retrieval and storage request to the database server 140 as required by the client application 115. As will be explained, this analysis may be extended to include event data from multiple patients 111.

The client device 110 communicates with the sensor 120 using a network adapter and either a wired or wireless communication protocol, an example of which is the Bluetooth Low Energy (BTLE) protocol. BTLE is a short-ranged, low-powered, protocol standard that transmits data wirelessly over radio links in short range wireless networks. After the sensor 120 and client device 110 have been paired with each other using a BTLE passkey, the sensor 120 automatically synchronizes and communicates information relating to medicament device usage with the client device 110. If the sensor 120 hasn't been paired with a client device 110 prior to a rescue medication event, the information is stored locally until such a pairing occurs. Upon pairing, the sensor 120 communicates any stored event records to the client device 110. In other implementations, other types of wireless connections are used (e.g., infrared or IEEE 802.11).

Although client devices 110 and medicament devices 160 are described above as being separate physical devices (such as smart phones and inhalers, respectively), the medicament devices 160 may include not only sensors 120 integrated into a single housing with the medicament device 160, but also aspects of the client device 110. For example, a medicament device 160 may include an audiovisual interface including a display or other lighting elements as well as speakers for presenting visual audible information. In such an implementation the medicament device 160 itself may present the contents of notifications provided by the server 130 directly, in place of or in addition to presenting them through the client devices 110.

The medicament device 160 is used to deliver medication to the lungs of a user experiencing a respiratory ailment rescue event. Different medicaments are used for asthma. Different medicaments may also be used for rescue and control for asthma. Medicament devices (e.g., inhalers) are typically portable and small enough to be carried by hand for ease of accessibility when treating respiratory attacks. In one embodiment, medication is delivered in aerosol form through a medicament device 160 such as a metered dose inhaler or a nebulizer. Metered dose inhalers included a pressured, propellant canister of aerosol medication, a metering valve for delivering a regulated medicine dosage amount, and a plastic holder that holds the pressurized canister and also forms a mouthpiece for delivery of the medication. In another embodiment, medication is delivered in dry powder form through a medicament device 160 such as a dry powder inhaler. Dry powder inhalers may have Cartesian ovular shaped bodies that house wheel and gear mechanisms enabling a user to index through a strip of dry powder medication. The bodies of dry powder inhalers also include a manifold and a mouthpiece to deliver dry powder to the user. Examples of controller medications that are dispensed by a controller medicament device 160 include Beclomethasone Dipropionate, Beclomethasone Dipropionate HFA Budesonide, Ciclesonide, Flunisolide, Fluticasone Furoate, Fluticasone Propionate, Mometasone, Mometasone furoate HFA 100 or 200 mcg, and Triamcinolone Acetonideas, as well as combinations of those medications with a long-acting bronchodilator such as salmeterol or formoterol. Other medications may include long-acting beta-agonists (LABA) such as Albuterol Sulfate, Formoterol Fumarate, Salmeterol Xinafoate, Arformoterol Tartrate, and Olodaterol. The long-acting beta-agonists may also include combinations such as Budesonide in combination with Formoterol; Fluticasone furoate, umeclidinium and vilanterol; Fluticasone Propionate and Salmeterol; Fluticasone furoate 100 mcg and Vilanterol 25 mcg; Glycopyrrolate/Formoterol Fumarate; Indacaterol/glycopyrrolate; Mometasone in combination with Formoterol; Tiotropium 2.5 mcg and Olodaterol 2.5 mcg; and Umeclidinium and Vilanterol. Examples of rescue medications that are dispensed by a rescue medicament device 160 include Albuterol Sulfate, Albuterol Sulfate HFA, Albuterol Sulfate inhalation and nebulizer solution, salbutamol, Levalbuterol HCl, metaproterenol, and terbutaline.

Each patient may be associated with more than one medicament device 160. For example, the patient may have a rescue medicament device 160 that dispenses rescue medication, and a controller medicament device 160 that dispenses controller medication. Similarly, each patient may be associated with more than one sensor 120, each chosen to operate with one of the patient's medicament devices 160.

Generally, a sensor 120 is a physical device that monitors the usage of the medicament dispenser 160. The sensor 120 is either removably attachable to the medicament dispenser without impeding the operation of the medication dispenser, or the sensor 120 is an integrated component that is a native part of the medicament dispenser 160 as made available by its manufacturer.

The sensor 120 includes its own network adapter (not shown) that communicates with the client device 110 either through a wired connection, or more typically through a wireless radio frequency connection. In one embodiment, the network adapter is a Bluetooth Low Energy (BTLE) wireless transmitter, however in other embodiments other types of wireless communication may be used (e.g., infrared, IEEE 802.11).

The sensor 120 may also be configured to communicate more directly with the application server 130. For example, if the network adapter of the sensor 120 is configured to communicate via a wireless standard such as IEEE 802.11 or LTE, the adapter may exchange data with a wireless access point such as a wireless router, which may in turn communicate with the application server 130 without necessarily involving the client device 110 in every exchange of data. These two methods of communicating are not mutually exclusive, and the sensor 120 may be configured to communicate with both the client device 110 and the application server 130, for example using redundant transmission to ensure event data arrives at the application server 130 or to provide information directly to the client device 110 while the application server 130 is determining what notification to provide in response to an event.

As introduced above, the sensor 120 captures data about usage of the medicament device 160. Specifically, each sensor 120 captures the time and geographical location of the rescue medication event, that is, usages of the rescue medicament device 160, by the patient 111. Each sensor 120 transmits the event data in real-time or as soon as a network connection is achieved, automatically without input from the patient 111, caregiver or healthcare provider 112. The medication event information is sent to the application server 130 for use in analysis, generation of asthma rescue event notifications, and in aggregate analyses of event data across multiple patients.

To accomplish this goal, there are a number of different ways for the sensor 120 to be constructed, and in part the construction will depend upon the construction of the medicament device 160 itself. Generally, all sensors 120 will include an onboard processor, persistent memory, and the network adapter described above, that together, function to record, store, and report medication event information to the client device 110 and/or server 130. Sensors 120 may also include a clock for recording the time and date of events.

Regarding specific constructions of the sensor 120, traditional inhalers, such as mechanical dose counters, are not designed with sensors 120 in mind, and thus the sensor 120 may be constructed accordingly. Some implementations in this manner include mechanical, electrical, or optical sensors to detect movement of the device 160, priming of the device, activation of the device, inhalation by the user, etc. In contrast, modern inhalers, such as deflectable membrane dose counters, include electrical circuitry may report event information as an electrical data signal which a sensor 120 is designed to receive and interpret, for example the medicament device 160 itself may report movement, priming, and activation to the sensor 120. Similar sensors may also be adapted for use with other types of medication application devices to detect and collect data on usage of such devices.

More information regarding hardware and software components for the sensors 120 and medicament devices 160, as well as the interaction between them to record rescue medication events can be found in U.S. patent application Ser. No. 12/348,424, filed Jan. 1, 2009, and International Application No. PCT/US2014/039014, filed May 21, 2014, both of which are incorporated by reference herein in their entirety.

The application server 130 is a computer or network of computers. Although a simplified example is illustrated in FIG. 2, typically the application server will be a server class system that uses powerful processors, large memory, and faster network components compared to a typical computing system used, for example, as a client device 110. The server typically has large secondary storage, for example, using a RAID (redundant array of independent disks) array and/or by establishing a relationship with an independent content delivery network (CDN) contracted to store, exchange and transmit data such as the asthma notifications contemplated above. Additionally, the computing system includes an operating system, for example, a UNIX operating system, LINUX operating system, or a WINDOWS operating system. The operating system manages the hardware and software resources of the application server 130 and also provides various services, for example, process management, input/output of data, management of peripheral devices, and so on. The operating system provides various functions for managing files stored on a device, for example, creating a new file, moving or copying files, transferring files to a remote system, and so on.

The application server 130 includes a software architecture for supporting access and use of the analytics system 100 by many different client devices 110 through network 150, and thus at a high level can be generally characterized as a cloud-based system. The application server 130 generally provides a platform for patients 111 and healthcare providers 112 to report data recorded by the sensors associated with their medicament devices 160 including both rescue medication and controller medication events, collaborate on respiratory ailment treatment plans, browse and obtain information relating to their condition and geographic location, and make use of a variety of other functions.

Generally, the application server 130 is designed to handle a wide variety of data. The application server 130 includes logical routines that perform a variety of functions including checking the validity of the incoming data, parsing and formatting the data if necessary, passing the processed data to a database server 140 for storage, and confirming that the database server 140 has been updated.

The application server 130 stores and manages data at least in part on a patient by patient basis. Towards this end, the application server 130 creates a patient profile for each user. The patient profile is a set of data that characterizes a patient 111 of a health care system such as a system directed toward addressing respiratory ailments. The patient profile may include identity information about the patient such as age, gender, current rescue medication, current controller medication, notification preferences, a controller medication adherence plan, a relevant medical history, and a list of non-patient users authorized to access to the patient profile. The profile may further specify a device identifier, such as a unique media access control (MAC) address identifying the one or more client devices 110 or sensors 120 authorized to submit data (such as controller and rescue medication events) for the patient.

The profile may specify which different types of notifications are provided to patients 111 or caregivers, coaches 113, and their personal healthcare providers 112, as well as the frequency with which notifications are provided. For example, a patient 111 or caregiver may authorize a healthcare provider 112 to receive notifications indicating a rescue event. The patient 111 or caregiver may also authorize their healthcare provider 112 be given access to their patient profile and rescue event history. If the healthcare provider 112 is provided access to the patient profile of the patient 111, the healthcare provider may specify controller adherence or rescue medication plans for the corresponding asthma condition. Medication plans may include a prescribed number of doses per day for controller medications.

The application server 130 also creates profiles for healthcare providers 112. A healthcare provider profile may include identifying information about the healthcare provider 112, such as the office location, qualifications and certifications, and so on. The healthcare provider profile also includes information about their patient population. The provider profile may include access to all of the profiles of that provider's patients, as well as derived data from those profiles such as aggregate demographic information, rescue and controller medication event patterns, and so on. This data may be further subdivided according to any type of data stored in the patient profiles, such as by geographic area (e.g., neighborhood, city) over by time period (e.g., weekly, monthly, or yearly).

The application server 130 receives rescue medication event information from the client device 110 or the sensor 120, triggering a variety of routines on the application server 130. In the example implementations described below, the data analysis module 131 executes routines to access respiratory ailment event data as well as other data including a patient's profile, analyze the data, and output the results of its analysis to both patients 111 or caregivers and providers 112. This process is generally referred to as a respiratory ailment risk analysis. In this example, the risk analysis is performed in relation to asthma for the patient 111. The asthma risk analysis may be performed at any point in time, in response to a rescue event, due to a relevant change in the patient's environment, and in response to any one of a number of triggering conditions discussed further below.

Other analyses are also possible. For example, a risk analysis may be performed on rescue and controller medication use for multiple patients to identify based on spatial/temporal clusters (or outbreaks) of medication use based on historically significant permutations from individual, geographic, clinical, epidemiologic, demographic, or spatial or temporal baselines or predicted or expected values. Other types of analyses may include daily/weekly adherence trends, adherence changes over time, adherence comparisons to other relevant populations (e.g., all patients, patients on a particular rescue medication or controller medication or combination thereof, identification of triggers (spatial, temporal, environmental), rescue use trends over time, and rescue use comparisons to other relevant populations. Thus, the percentage of adherence for a particular patient is calculated by the number of controller puffs that are taken on a specific day divided by the daily number of puffs that are prescribed. This daily metric is then averaged over a specified time period to get the mean daily adherence. The number of controller puffs prescribed is based on the patients' medication plan, which is entered into the application.

Responsive to any analyses performed, the application server 130 prepares and delivers push notifications to send to patients 111, authorized healthcare providers 112, and/or other users provided access to the patient's profile, such as caregivers. Notifications can provide details about the timing, location, and affected patient(s) 111 involved in a medication rescue event. Notifications may additionally comprise a distress or emergency signal that requests emergency assistance that are distributed to emergency assistance providers 112. Notifications may also include the results of the asthma risk analysis performed by the data analysis module 131. More information regarding the types of notifications that may be sent and the content they may contain is further described below.

In addition to providing push notifications in response to an asthma risk analysis, notifications may also be provided as pull notifications, at particular time intervals. Additionally, some notifications (whether push or pull) may be triggered not in response to an asthma risk analysis performed in response to a rescue medication event, but instead in response to a risk analysis performed in response to one of the underlying factors in the asthma risk analysis changing, such that an updated notification is warranted. For example, if weather conditions indicate that an increase in air pollution is occurring or is imminent, this may trigger conducting asthma risk analyses for all patients located in the particular geographic area where the pollution is occurring.

Notifications are provided through the network 150 to client applications 115 in a data format specifically designed for use with the client applications, and additionally or alternatively may be provided as short message service (SMS) messages, emails, phone calls, or in other data formats communicated using other communication mediums.

The database server 140 stores patient and provider data related data such as profiles, medication events, patient medical history (e.g., electronic medical records). Patient and provider data are encrypted for security and is at least password protected and otherwise secured to meet all Health Insurance Portability and Accountability Act (HIPAA) requirements. Any analyses (e.g., asthma risk analyses) that incorporate data from multiple patients (e.g., aggregate rescue medication event data) and are provided to users is de-identified so that personally identifying information is removed to protect patient privacy.

The database server 140 also stores non-patient data used in respiratory ailment risk analyses. In this example, the data may be relevant for asthma or COPD. This data includes regional data about a number of geographic regions such as public spaces in residential or commercial zones where patients are physically located and may be exposed to pollutants. This data may specifically include or be processed to obtain a patient's proximity to green space (areas including concentrated numbers of trees and plants). One example of regional data includes georeferenced weather data, such as temperature, wind patterns, humidity, the visibility, and so on. Another example is georeferenced pollen or pollution data, including concentrations for all criteria pollutants modeled at an instance of time and/or measured empirically. Additional assessments of patient residential or work neighborhood characteristics that are relevant to respiratory outcomes may also be included. Examples include: an assessment of socioeconomic (e.g., income, education, percent home ownership) and demographic factors (e.g., race/ethnicity, age, language and gender composition) derived from census data and other data sources. Additional evaluations of the residential and/or work environments may also be included, such as evaluation of the built environment (e.g., walkability, crime, proximity to major roadways or other pollution sources). All of the data above may vary over space and time, and as such the data itself may be indexed by time, for example separate data points may be available by time of day (including by minute or hour), or over longer periods such as by day, week, month, or season. Although the database server 140 is illustrated in FIG. 1 as being an entity separate from the application server 130, the database server 140 may alternatively be a hardware component that is part of another server such as server 130, such that the database server 140 is implemented as one or more persistent storage devices, with the software application layer for interfacing with the stored data in the database is a part of that other server 130.

The database server 140 stores data according to defined database schemas. Typically, data storage schemas across different data sources vary significantly even when storing the same type of data including cloud application event logs and log metrics, due to implementation differences in the underlying database structure. The database server 140 may also store different types of data such as structured data, unstructured data, or semi-structured data. Data in the database server 140 may be associated with patients, groups of patients, and/or entities. The database server 140 provides support for database queries in a query language (e.g., SQL for relational databases, JSON NoSQL databases, etc.) for specifying instructions to manage database objects represented by the database server 140, read information from the database server 140, or write to the database server 140.

The network 150 represents the various wired and wireless communication pathways between the client 110 devices, the sensor 120, the application server 130, and the database server 140. The network 150 uses standard Internet communications technologies and/or protocols. Thus, the network 150 can include links using technologies such as Ethernet, IEEE 802.11, integrated services digital network (ISDN), asynchronous transfer mode (ATM), etc. Similarly, the networking protocols used on the network 150 can include the transmission control protocol/Internet protocol (TCP/IP), the hypertext transport protocol (HTTP), the simple mail transfer protocol (SMTP), the file transfer protocol (FTP), etc. The data exchanged over the network 150 can be represented using technologies and/or formats including the hypertext markup language (HTML), the extensible markup language (XML), etc. In addition, all or some links can be encrypted using conventional encryption technologies such as the secure sockets layer (SSL), Secure HTTP (HTTPS) and/or virtual private networks (VPNs). In another embodiment, the entities can use custom and/or dedicated data communications technologies instead of, or in addition to, the ones described above.

FIG. 2 is a high-level block diagram illustrating physical components of an example computer 200 that may be used as part of a client device 110, application server 130, and/or database server 140 from FIG. 1, according to one embodiment. Illustrated is a chipset 210 coupled to at least one processor 205. Coupled to the chipset 210 is volatile memory 215, a network adapter 220, an input/output (I/O) device(s) 225, a storage device 230 representing a non-volatile memory, and a display 235. In one embodiment, the functionality of the chipset 210 is provided by a memory controller 211 and an I/O controller 212. In another embodiment, the memory 215 is coupled directly to the processor 205 instead of the chipset 210. In some embodiments, memory 215 includes high-speed random access memory (RAM), such as DRAM, SRAM, DDR RAM or other random access solid state memory devices.

The storage device 230 is any non-transitory computer-readable storage medium, such as a hard drive, compact disk read-only memory (CD-ROM), DVD, or a solid-state memory device. The memory 215 holds instructions and data used by the processor 205. The I/O device 225 may be a touch input surface (capacitive or otherwise), a mouse, track ball, or other type of pointing device, a keyboard, or another form of input device. The display 235 displays images and other information from the computer 200. The network adapter 220 couples the computer 200 to the network 150.

As is known in the art, a computer 200 can have different and/or other components than those shown in FIG. 2. In addition, the computer 200 can lack certain illustrated components. In one embodiment, a computer 200 acting as server 140 may lack a dedicated I/O device 225, and/or display 218. Moreover, the storage device 230 can be local and/or remote from the computer 200 (such as embodied within a storage area network (SAN)), and, in one embodiment, the storage device 230 is not a CD-ROM device or a DVD device.

Generally, the exact physical components used in a client device 110 will vary in size, power requirements, and performance from those used in the application server 130 and the database server 140. For example, client devices 110, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out the asthma risk analysis introduced above. In one embodiment, the processing power of the application server 130 provided by a service such as Amazon Web Services™. Also, in contrast, the database server 140 may include many, physically separate computers each having a significant amount of persistent storage capacity for storing the data associated with the application server.

As is known in the art, the computer 200 is adapted to execute computer program modules for providing functionality described herein. A module can be implemented in hardware, firmware, and/or software. In one embodiment, program modules are stored on the storage device 230, loaded into the memory 215, and executed by the processor 205.

In this example, an application executed on the application server 130 may collect active data such as use data from medicament devices such as the device 160 in FIG. 1. Additionally, context data such as location, environmental condition may be collected. The application server 130 also accesses historical data relating to a relevant patient population for recommendation of changes in treatment regimen. The application server 130 collects data from uses of medicament devices by a patient population that can be used with other patient data to provide predictions for stepping up or stepping down treatments for respiratory ailments. For example, the treatments may be different inhaler medications according to GINA guidelines on asthma treatments. Other more advanced treatments such as biologics may be used in the final step of the asthma treatment. Such suitable biologic treatments may include for example omalizumab (Xolair), mepolizumab (Nucala), reslizumab (Cinqair), benralizumab (Fasenra), and dupilumab (Dupixent) for the most extreme treatment step.

For example, collected medication and inhaler usage data may be used to determine factors such as: a) use of inhaled corticosteroids (ICS) or bronchodilator medicine such as LABA; b) proof of use of ICS/LABA; and c) proof of controller adherence. Additional inhaled corticosteroids data relating to asthma control may be analyzed. Such data relating to asthma may include rescue inhaler usage, nighttime symptoms, an asthma control score (ACT), asthma control by ACT, and external sensor readings such as spirometry readings.

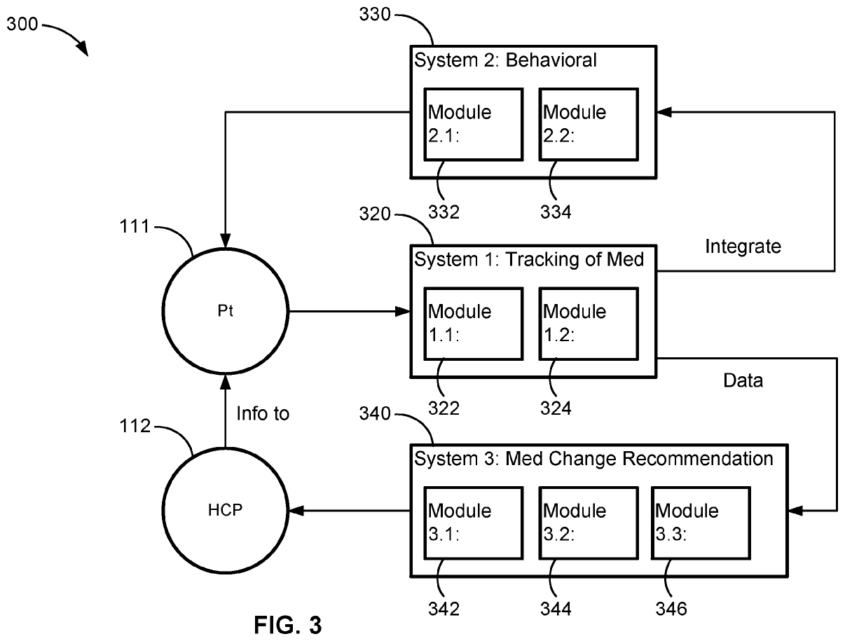

FIG. 3 is a block diagram of a health care collection and clinical decision support system 300 that includes different information collection and analysis systems that determine appropriate step-up or step-down of treatments for the patient 111. This information is provided to a healthcare professional 112. The support system 300 includes a medication tracking system 320, a behavioral data tracking system 330, and a medication change recommendation system 340. The medication tracking system 320 includes a passive capture module 322 and a categorization module 324. The passive capture module 322 captures patient medication usages and reports the dates, times, and locations back to the broader system 320. Thus, the passive capture module 322 interfaces with the sensor 120 in FIG. 1 in this example for inhaler based medication. Other mechanisms may be used to collect usage data for inhaler based medication or other types of medications such as patient reporting, container based data, medication supply chain fill data, or measurements from other sensors such as the patient worn body sensor.

The categorization module 324 categorizes whether a rescue puff from the inhaler 160 was preemptive or an emergency use based on the collected data. The categorization module 324 categorizes whether the dose was taken properly (i.e., with correct inhaler technique). The categorization module 324 is part of the application on the user device 110 that includes data input from GPS location, and sensor data on flow rate or positioning. The application also includes interface generation for receiving patient input such as a survey or questionnaire that may be displayed to the patient at predetermined times.

The behavioral tracking system 330 includes an adherence support module 332 and a symptom support module 334. The adherence support module 332 provides an application executed by the client device 110 such as a mobile device for focused interventions to motivate behavior change on the behalf of the patient. Thus, the adherence support module 332 may employ strategies such as displaying treatment information or providing motivation strategies such as gamification software or incentives, to improve adherence. Thus, if poor adherence is due to inhaler technique, the application may focus education here, such as videos, coaching or surveys on technique questions through the client device 110. Inhaler technique may be evaluated through inhaler timing and technique based on sensor data collection. Instructions to remedy deficient techniques may be displayed by the application. The symptom support module 334 focuses on behavior change focused interventions, e.g., education, to improve patients' symptoms. Other relevant data such as side effects, barriers to treatment, perceptions of medication, may also be collected by the symptom support module 334. Such data may be collected through patient surveys, EHR, insurance data, or proxy data based on location or other patient demographic information.

The system 300 uses passive patient medication tracking to initiate behavior interventions directly to the patient to improve clinical outcomes (e.g., medication adherence). It further leverages the data to offer clinical decision support. Specifically, the system captures the medication usage information relating to the patient and applies smart determinations to categorize that usage.

For example, the determinations may include whether rescue usage of the inhaler was preventive or emergency usage. Another determination is adherence to proper inhaler technique. Based on the rescue usage data, the system 300 may make a medication change recommendation to the healthcare provider 112, with a justification for that recommendation.

Timing for issuing recommendations of the change is treatment may be automatic. For example, the timing may be based on either: timing of a visit of the patient 111 to the healthcare provider 112, treatment change based on need as a result of an acute worsening, or according to guideline recommendations for medication review (e.g., 2-3 months after a change in treatment). The systems of the support system 300 represents the collected data in an exportable report that may support documentation for medication changes, such as prior authorization forms required for some specialty drugs (e.g., biologics). Thus, the collected data may simply be provided to the healthcare provider 112 without the recommendation output of the medication change recommendation system 340. The collected data is captured and structured in a visual way, allowing better decision making by a healthcare provider reviewing the structured data. Alternatively, a recommendation may be provided automatically based on the structured data to the healthcare provider 112. Of course, both the recommendation as well as the underlying data may be provided to the healthcare provider 112.

The medical change recommendation system 340 includes a change recommendation module 342, a notification module 344, and a document management module 346. The recommendation module 342 is a data analysis engine that determines a patient value associated with the treatment regimen, makes a comparison of the patient value to a threshold level, and provides a recommendation to change the step of the treatment regimen based on the comparison to the threshold level. The recommendation module 342 executes a machine learning based algorithm to determine whether a change in medication should be made. As will be explained below, the algorithm may be rule based and include customizable thresholds, if simple. The rules-based version determines thresholds for step-up or step-down recommendations from machine learning analysis. In this version, thresholds may be “over-ruled” by a healthcare professional. Another version is machine learning based recommendations without customizable thresholds.

The machine learning model produces risk score (i.e., from 0-1 or 0-100) relating to step-up or step-down decisions. The risk score alone could be made available to a healthcare professional to make the step-up/step-down decision. However, a threshold level for the different step-up or step-down could be made for different ranges of risk scores to automate the decision to recommend a step-up or a step-down in treatment. The automated decision based on the threshold levels replaces current subjective decisions by health care providers.

The different decisions may thus be scored as probabilities across all choices that sum to 1 or 100, in which case the decision or “threshold” is really just the maximum score of the three buckets (e.g., if it was 20% step-down, 70% maintain, 10% step-up, the recommendation may be to maintain).

Similarly, a more nuanced decision system that recommends specific drugs and doses, may be based on some kind of set of scores or probability distributions, which can be simplified/abstracted away if desirable. For example, the model might recommend 3 drugs with positive probabilities and the rest 0%, and of those 3 they might be 70/20/10. And then each of those drugs have an associated probability distribution of the right dosage method and level.

The recommendation output is based on classification of whether a medicament device activation was for rescue or controller use. The classification occurs earlier in the process, in the data gathering phase. The classification may be a manual, user label input, or it could be rule based, or it could utilize a separate machine learning classification routine. It is desirable to select the classification method to accurately gather data. Based on the categorization of the medication categorization, it may be determined whether a rescue medication was pre-emptive or whether a controller medication had proper technique. When considering a step-up/step-down recommendation, the machine learning model may look at all rescue and all controller puffs, regardless of categorization. Alternatively, the model may consider only the rescue uses that were emergency and controller uses that were “properly taken.”

The tiers or steps of treatment are specific to the particular ailment. For example, asthma may be addressed through a multi-step treatment regimen as recommended by GINA (https://ginasthma.org/wp-content/uploads/2020/04/Main-pocket-guide_2020_04_03-final-wms.pdf, pp. 22-28) depending on the age of the patient. At the current time, the first step for adults includes a preferred controller that may be a low dose inhaled corticosteroid (ICS)-formoterol with an optional ICS whenever SABA is taken. The preferred reliever of the first step is an as-needed low dose ICS-formoterol with an optional reliever being an as-needed short-acting Beta-agonist (SABA). The second step treatment is a daily low dose inhaled corticosteroid (ICS) or as-needed low dose ICS-formoterol. An alternate control is a daily leukotriene receptor antagonist (LTRA), or low dose ICS taken whenever SABA is taken. The preferred reliever of the second step is an as-needed low dose ICS-formoterol with an optional reliever being an as-needed short-acting Beta-agonist (SABA). The third step includes a preferred controller that is low dose ICS-LABA (long-acting beta-agonist). An alternative controller is a medium dose ICS, or a low dose ICS+LTRA. The preferred reliever of the third step is an as-needed low dose ICS-formoterol for patients prescribed maintenance and reliever therapy with an optional reliever being an as-needed short-acting Beta-agonist (SABA). The fourth step includes a preferred controller that is a medium dose ICS-LABA. An alternative controller is a high dose ICS, add-on tiotropium or add-on LTRA. The preferred reliever of the fourth step is an as-needed low dose ICS-formoterol for patients prescribed maintenance and reliever therapy with an optional reliever being an as-needed short-acting Beta-agonist (SABA). The fifth step includes a controller that is a high dose ICS-LABA. A reference may be made for phenotypic assessment and add-on therapy such as tiotropium, anti-IgE, anti-IL5/5R or anti-IL4R. An alternate controller may be a low dose OCS. The preferred reliever of the fifth step is an as-needed low dose ICS-formoterol for patients prescribed maintenance and reliever therapy with an optional reliever being an as-needed short-acting Beta-agonist (SABA). These guidelines and recommended treatment steps are reviewed and adjusted as needed on a regular basis by guidelines committees. The system described here would be reviewed regularly and amended to adhere to the most current guidelines as they are released. Other ailments and diseases with similar treatments having different steps may incorporate the principles incorporated herein for changes to different medications in the different steps.

Thus, the machine learning algorithm in this example determines whether changes between the steps of the GINA treatment regimen are required based on the collected data. The output may thus recommend either a step-up, a step-down, or a maintenance of the current step based on the input of use data, and review of historical trends and patient contextual data. Another example of a multi-step asthma treatment regimen is the NHLBI NAEPPCC. The system thus enhances treatment of respiratory ailments such as asthma by the accurate prediction of an appropriate treatment.

The notification module 344 alerts the healthcare provider 112 about the system recommendation for medication management in an on-screen alert or inbox notification on a mobile device or other computer. The notification module 344 provides an interface that allows the display of the profile of patients to the healthcare provider 112. The interface highlights the potential for a medicament management shift within the profile. Another interface provides notification based on the output of a provider notification system. The notification system includes timing logic to provide recommendations for changes in treatment at a given point of time viewed as well as during a given interval determined by guideline recommendation for medication review (e.g., 3 months after change).

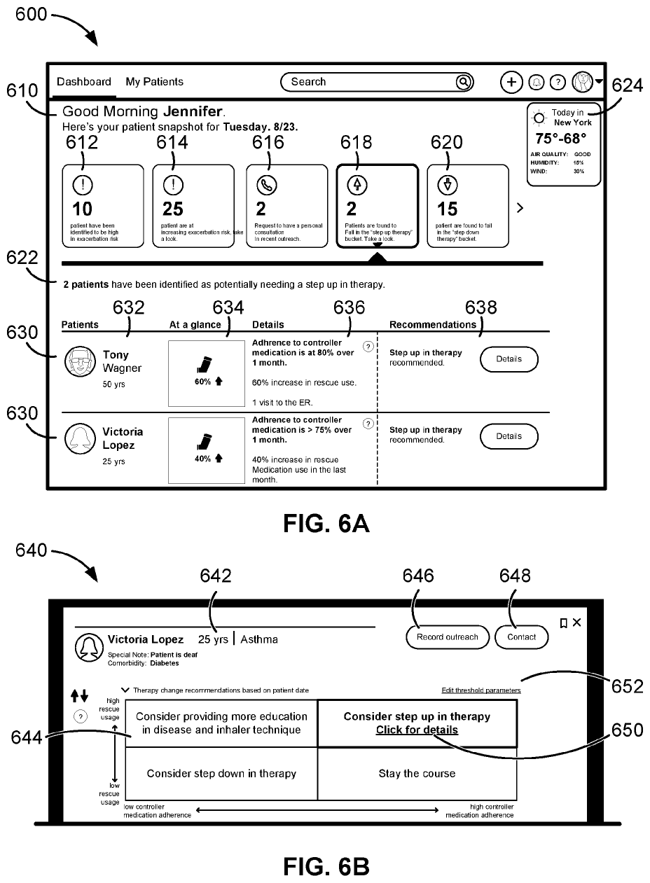

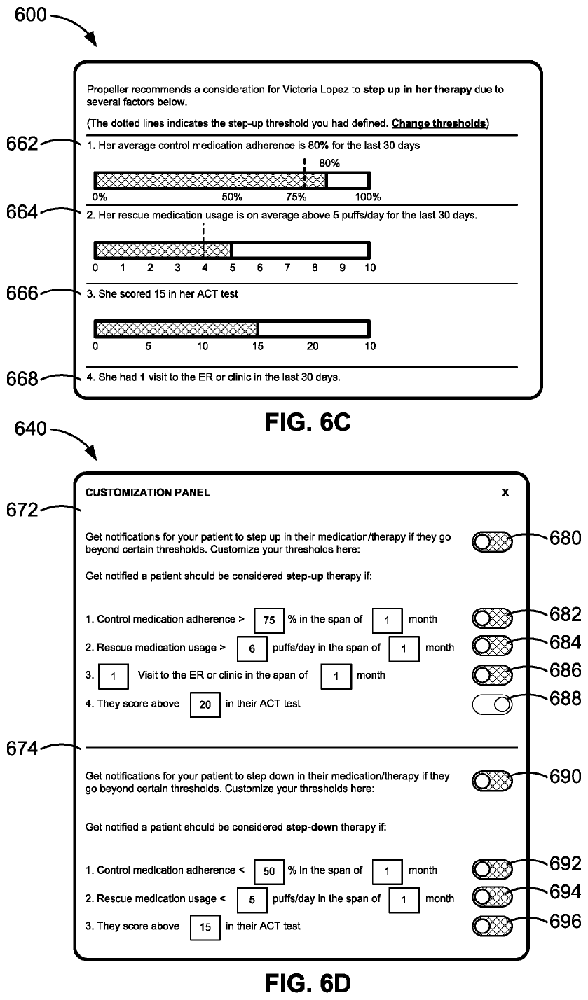

The document management module 346 produces reports representing the collected data. The reports may include a 2x2 chart and associated options selected. An example 2x2 chart for rescue use and adherence thresholds to determined classification is shown in FIG. 6B. The document management module 346 may include data provided in a medication history format such as a timeline. Other data outputs may include graphics reflecting adherence (correct versus incorrect usage) and type of rescue (preventive versus emergency). The document management module 346 may also provide the data for interface that provides medication history and plans for a patient. Such an interface, may put the current recommendation in context of the prior patient history.

The passive capture module 322 may be extended to include more forms of data collection that may be relevant for making determinations of changes in treatment regimens. For example, the passive capture module 322 may include data interfaces specific to data collection from other devices. This may include additional passive collection of patient metrics through hardware such as Fitness trackers (activity metrics, such as steps walked, elevation, heart rate). Pulse Oximeters (PO2, respiratory rate, Oxygen saturation), and night time monitoring (respiratory rate, awakeness/night time symptoms through devices such as fitness trackers or smart watches) The passive capture module 322 may also include additional passive collection of metrics through data aggregator software such as the Apple Health Kit, Google Fit and/or EHR based applications. The passive capture module 322 may include functionality for additional active collection of metrics through other medical based hardware or software. This may include spirometry through a connected device via Bluetooth or determining Peak Flow, via Bluetooth, physical activity and other physiological signals through a smartwatch and other devices or through photo capture in conjunction with a digital spirometer application.

The passive data capture module 322 may also be extended to include additional active collection of patient data through survey interfaces generated by the module on the computer device 110. These surveys may be directed toward symptomatic status or quality of life. The surveys may include clinical surveys on disease status such as the ACT or the COPD Assessment Test (CAT). Another survey may be geared toward exacerbation history. The exacerbation history could be taken during enrollment (12-month history), as a monthly input in the application, as tracking as an event in a calendar or through location identification (i.e., presence at a medical care facility). The historical data may also be pulled from claims data or EHRs with the appropriate data interfaces to the passive data capture module 322.

Another survey may be for determining comorbidities that may be deployed in an interface generated by the application in the mobile device. The comorbidities based data may also be pulled from a connected EHR (ICD-10 codes & medications) including allergic rhinitis, eczema, food allergy, and obesity data.

Another survey may be for determining tobacco use or exposure. The tobacco data may be gathered via an interface generated by the application on the mobile device. This data may be extended to support and supplement behavior change programs, education, or referral to a tobacco cessation program. Similar surveys may be directed to other detrimental substances.

Environmental risk factors may be determined by surveys, self-reported triggers and symptoms in the application, as well as objective assessments of environmental exposure and sensitivity based on an objectively-collected location. Such locations could include: a residence, a workplace, user phone location, sensor sync locations and/or medication use locations. Each unique location of interest is assigned an environmental exposure signature, including multiple air pollutants, weather variables, pollen, and built environment factors (e.g., proximity to major roadways or other pollution emission sites). The survey about indoor allergen exposures can determine exposures at home and work.

Allergies surveys may include data from EHRs and lab results (e.g., allergen testing). Other surveys provided through the computing devices may allow patients and/or caregivers to set goals as part of early enrollment process. For example, each patient or caretaker could set personal self-management goals for their treatment, such as activities they may want to achieve such as: walking 1 mile, playing football with grandkids, or being able to do their shopping at the store. The system collects this information, display it and track and reports progress against the goal over time. These surveys could also explore additional factors that may influence medication-taking behavior and regimen adherence, such as perceptions of medication risk or effectiveness, side effects, financial barriers to adherence, or other barriers. All of this information would be incorporated into a shared decision-making framework for the clinical provider and patient to discuss when making treatment decisions. A shared decision-making framework implemented for clinical practice may entail elements of: 1) patient and provider goal setting; 2) discussion of the reasonable treatment options available; 3) a discussion of details related to those options and the relative benefits and drawbacks; and 4) discussion to support the work of decision-making and coming to the best conclusion.

The application allows a review of medication history, including medication taking behavior and any adjustments to regimen. Data collection may include surveys including medication data from connected EHRs or pharmacy records, a longitudinal review of all medication taking behavior collected with the sensors, and a survey that asks a patient about their medication history.

The module 342 may also be used to store any applicable action plans. [For example, an asthma action plan may be referenced via an image captured of the plan provided from a clinician. Alternatively, such a plan may be electronically transmitted. The information from the action plan may be used to align the action plan with a medication plan.

The passive data capture module 322 may include an evaluation of social determinants of health (SDoH) summary. According to the World Health Organization, SDoH are the conditions in which people are born, grow, live, work and age. These circumstances are shaped by the distribution of money, power, and resources at global, national, and local levels. Collectively, the SDoH have a much larger impact on the health outcomes for an individual than does medical care. The system will collect relevant SDoH information that: 1) may influence health outcomes for the patient; and 2) may be relevant or recommended by clinical guidelines to address when considering a step-up or step-down in treatment. A SDoH evaluation may include information capture of various SDoH factors including but not limited to: physical environment (e.g., indoor or outdoor environmental exposure, housing type or quality, built environment characteristics, transit access, walkability, crime, proximity to major roadways or other pollution sources), social and economic factors (e.g., education, employment, income, social support, safety), and health behaviors (e.g., tobacco use, diet and exercise, alcohol and drug use). Factors may also include urban—rural commuting area (RUCA), food access, racial isolation, redlining, social vulnerability assessments (e.g., Social Vulnerability Index, County Health Rankings, Area Deprivation Index), small area estimates on chronic disease-related health risk behaviors, health outcomes, health status, use of preventive services, clinical care, length and quality of life, physical environment and policy, broadband access and access to health care. The environmental exposure for a patient may be assessed using sensor/heartbeat and medication use locations (as well as residential or work addresses) to quantify air pollution and other environmental exposure, survey about indoor allergen exposures at home and work, and by identifying community-wide factors that influence respiratory health, including but not limited to air quality, traffic, built environment factors and urban heat. Screening for housing issues can conducted by survey, and housing quality and age may be assessed by publicly available property information. Assessment of socioeconomic (e.g., income, education, percent home ownership), food and transit access, and demographic factors (e.g., race/ethnicity, age, language and gender composition) can be collected via surveys, or derived from census data and other data sources assigned to the residential address. Additional health behaviors and comorbidity prevalence (e.g., diabetes or being overweight) can be collected via surveys, or via neighborhood-level prevalence available via national survey data.

The SDoH data may be summarized and shared with other stakeholders, such as SDoH platforms, a health system's population health management program, or social workers. The SDoH data allows the creation of the context for why a patient might have high symptoms or low adherence, adding a level of context to the technological solution of the clinical support system 300. This data may therefore be incorporated in the recommendation by providing information for SDoH that may require a step-up in treatment.

The notification module 344 may be extended to include support for escalation pathways to the steps of the treatment regimen. Such pathways would be provided to healthcare providers via a portal. For example, a notification of sending an ACT survey may be displayed to a healthcare provider. The healthcare provider may then select an option to send the ACT survey to obtain relevant information to support step-up or step-down action. For example, the healthcare provider 112 may push a request for more patient reported information instead of constant application inputs, as such inputs may be provided at the discretion of a healthcare provider as part of an evaluation of the patient. Another escalation pathway may be based on the healthcare provider pulling data from an EHR or other relevant information into the system, such as medication history. Another pathway is a connection to a prior authorization system to call out needed information for a step-up action. This pathway would generate the requirements as requests to the patient or format current information to support the step-up in treatment. Another pathway is a connection to a formulary related to the patient to recommend specific medicine that could be tried instead of the current medication.

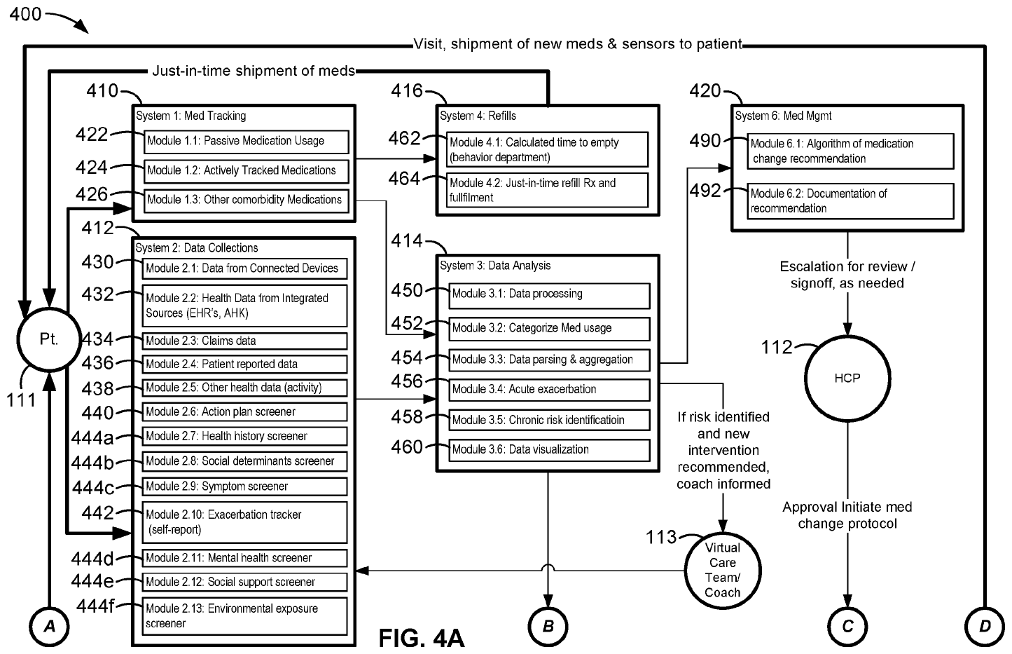

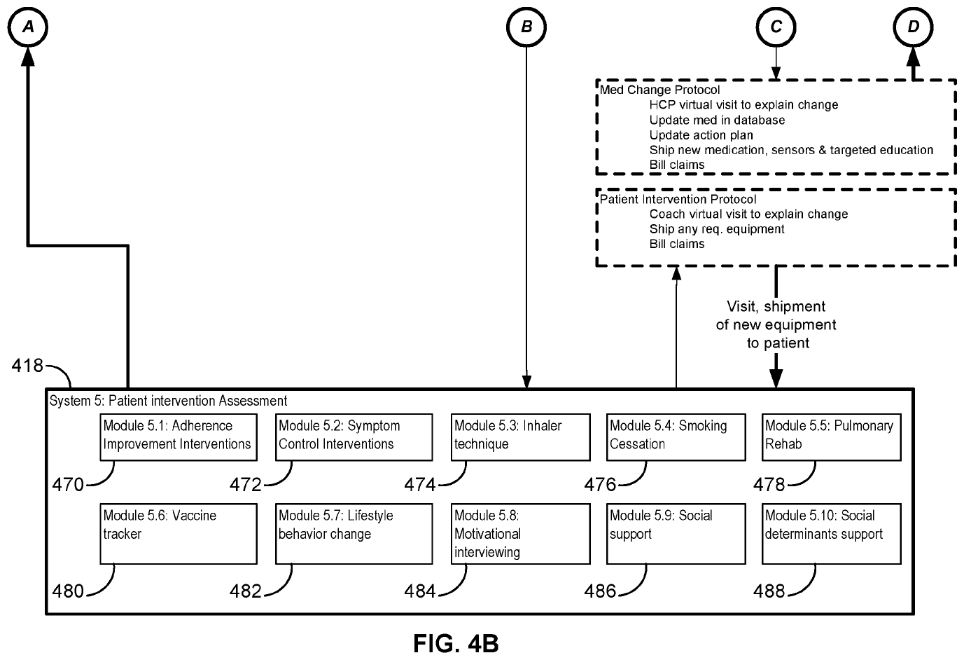

FIGS. 4A-4B are a diagram of a full virtual medication management and recommendation system 400 that provides automatic management of treatment for a patient. The system 400 includes a medication tracking system 410, a data collection system 412, a data analysis system 414, a refills system 416, a patient intervention assessment system 418, and a medication management system 420. The recommendation system 400 provides step-up or step-down actions for the patient 111 that has been prescribed as a treatment regimen from the healthcare provider 112. Additional human resources such as the coach 113 may use the analysis provided by the system 400.

The system 400 is designed to track a patient's usage of medication. The system 400 collects health-information from the patient, both at enrollment and on an ongoing basis. The system 400 analyzes the data collected to send medication refills directly to the patient, initiate patient interventions, as needed, and change a patient's medication regimen based on the analysis. Further the data may be used to enable analysis and risk identification for patients.

The medication tracking system 410 includes a passive medication usage module 422, an active tracking module 424, and a comorbidity medication module 426. Both modules 422 and 424 receive data from the sensor 120 through the application on the computer device 110. The passive capture module 422 may also collect usages for purposes of adherence in relation to inhaler uses. Inhaler-based medication may include controller medication or rescue medication. Other mechanisms may be used to collect usage data for inhaler-based medication or other types of medications such as patient reporting, container based data, medication supply chain fill data, or measurements from other sensors such as the patient worn body sensor. Thus, the principles disclosed herein are not limited to the example inhaler based medication. For example, other medications may include SMART/MART therapy, nebulizer-based medication, injectables, oral medications, allergy medications, and other medications for comorbidities. Certain devices such as nebulizers and injectors may be tracked with passive sensors integrated with the nebulizer or injector or attached to the device. The passive medication module 420 captures patient medication usage and reports the dates and times of the usage to the broader system 400. The active tracking module 424 is an application on the mobile device 110 associated with the patient 111 that has medication tracking functions. The active tracking module 424 includes an interface allowing for manual entry of any relevant respiratory medication such allergy medications or oral medication such as oral steroids. The comorbidity module 426 may receive data from other databases relating to relevant comorbidity medications taken by the patient.

The data collection system 412 includes a device module 430, a health data record module 432, a claims screener 434, a patient reported data module 436, and a health data module 438, an action plan checker 440, and an exacerbation tracker 442. The data collection system 412 also includes a series of screeners including a patient health history screener 444a, a SDoH screening tool 444b, a symptom screener 444c, a mental health screener 444d, a social support screener 444e, and an environmental exposure screener 444f.

The device module 430 integrates data for all applicable connected devices that a patient has linked into the system. For example, these devices may be the mobile device 110, a fitness tracker, smart watch, digital spirometers, pulse oximeters or other medical-grade Bluetooth devices. The integrated health data record module 432 integrates data from other sources of health data such as medical records (e.g., EHRs, or pharmacy data) or other data aggregators (e.g., Apple Health Kit). The integrated health data record module 432 uses software connection between individual databases and therefore has specific APIs for interfacing the databases to the module 432. The data record module 432 may receive daily feeds from databases such as an EHR database.

The claims data module 434 may access different payer databases or a claims data aggregator source. The claims data collected by the claims data module 434 may be used to collect or supplement patient related health data. Claims data may include procedures, comorbidities, acute care utilization: emergency department visits, hospitalizations, other health care utilization such as office visits, pharmacy fill data, and cost data. The claims data may be used to confirm EMR data or supplement the data for completeness of a patient data record.

The patient reported data module 436 includes interfaces that allows questions asking patients directly about their disease, such as symptomatic status or quality of life, as well as clinical surveys on disease status such as the ACT or CAT, etc. The application may be set to display surveys at times such as on a regular schedule (once a week), event-based (after taking a set number of rescue medications) or on request by the healthcare provider 112 or the coach 113. The additional health data module 438 allows collection of additional health related data as described above. Such additional information may include any data that influences health, such as the social determinants of health explained above. The action plan check module 440 checks to determine whether a patient has an action plan for what to do when symptoms arise. The action plan check module 440 tracks the action plans and any updates due to changes in the treatment protocol or medications. The exacerbation tracker module 442 collects data input by a patient relating to exacerbation, for example through questions displayed on an interface.

The patient health history screener 444a may collect patient variables during enrollment and during the period of treatment. The patient health history screener 444a may collect data via the application on a user device, through SMS, or the application card as described above. The patient variables may include exacerbations over the previous 12 months, COPD diagnosis and the time since diagnosis, comorbidities, smoking history, relevant demographics (age, gender, race, ethnicity, self-reported income, education), and body mass index (kg/m2).

The SDoH screening tool 444b may determine social determinants based on the address of the user. The data may be derived at the county/census tract/census block level. Example social determinants that may be derived include urban—rural commuting area (RUCA), food access, racial isolation, redlining, social vulnerability assessments (e.g., Social Vulnerability Index, County Health Rankings, Area Deprivation Index), small area estimates on chronic disease-related health risk behaviors, health outcomes, health status, use of preventive services, clinical care, length and quality of life, physical environment and policy, broadband access and access to health care.

The symptom screener 444c collects data from symptom tests such as CAT or ACT. The symptom screener 444c may also enable analysis of changes in test results over time. The symptom screener 444c may also collect other types of symptom related data such as recording coughs of a user. The screener 444c may interface with a user device to record detected coughs or provide an interface to ask a user to record a cough or other sounds. The screener 444c may also collect data relate to a quality of life or frailty index.

The mental health screener 444d collects data from the answers to self-reported questions such as those in the PHQ 2/9 or GAD7. The mental health screener 444d may also collect related data such as communication activity data (e.g., the number, frequency and timing of phone or text contacts, or deviations from normal patterns), physical activity, and mobility assessment determined by GPS coordinates reported by a user device. The mobility assessment may be based on distance assessments including but not limited to: total distance traveled, radius of area traveled, minimum convex polygon or time away from home. The communication activity data may include the number of frequency, and time of day of texts and messages versus a normal base line, and number, frequency time of day, and length of calls against a normal baseline.

The social support screener 444e collects data related to social support. The data may include inputs from a caregiver through a questionnaire or report. The data may include social media account linking and assessment of usage of the media account, as well as the depth and breadth of the social network. Other communication data such as phone or text message activity may be evaluated similar to the mental health screener 444d.

The environmental exposure screener 444f collects daily environmental data such as air pollution, and weather data based on the location of the patient. The environmental exposure screener 444f also collects data related to chronic exposure based on the home address of the patient. Data such as airborne toxics such as from the EPA National Emissions Inventory (NEI), or Air Toxics Screening Assessment (AirToxScreen) and other substances in the area of the home address may be collected. Data relating to an environmental justice analysis such as the Environmental Justice Screening and Mapping Tool may also be collected by the environmental exposure screener 444f.

The data analysis system 414 includes a data processing module 450, a medication categorization module 452, a data parsing and aggregation module 454, an exacerbation risk notification system 456, a chronic risk identification module 458, and a data visualization module 460. The data processing module 450 first merges and links the historical data collected through various sources before the data is de-identified in the data lake, thereby reducing the amount of training data needing to be processed. The data processing module 450 then curates an exhaustive list of variables from the de-identified data to generate and append to a denormalized table. The curation process may include deduplication as well as logics to rank what data source to use for certain variables that can be sourced from two or more sources for the same patient. The deduplication in the curation process reduces the amount of training data needing to be processed, while the ranking ensures that the most accurate data is used to train the model. In addition, the ranking allows for flexibility in the scenario that the highest ranked source may not contain all necessary data, and indicates which data source can also be trusted to provide data that does not compromise the accuracy of the model. Each row of the denormalized table represents a single day of a single patient. All of the variables are assigned a column in the denormalized table. Any variables that are not available for a given patient-day are assigned a null value.

The medication categorization module 452 categorizes the collected data relating to medication. For example, when receiving rescue data, the module 452 categorizes whether a puff was preemptive or emergency. When receiving controller data, the module 452 categorizes whether the dose was taken properly (i.e. with correct inhaler technique). The module includes software logic that leverages different background data such as GPS location, inputs from the patient (e.g., a survey answer that “I used this preemptively”), and sensor data on flow rate or positioning from the sensor 120.

The data parsing and aggregation module 454 considers the data points from the various sources within the medication tracking system 410 and unifies them or verifies them. For example, if there are two devices that measure respiratory rate, the aggregation module 454 will determine which respiratory rate value is more reliable and accurate. The data parsing and aggregation module 454 uses software logic that weighs the verity of individual hardware signals (e.g., a medical device in relation to a wellness tool) to select the “dominant” signal between devices that measure the same metric and therefore increases efficiency of computing resources by reducing the considered data. The data visualization module 454 allows the display of interfaces that show the collected data in different formats such as a graph, graphics, trend lines and the like.

The exacerbation risk notification system 456 provides warning for exacerbation risk to the patient, caregiver, coach and/or health care professional. The exacerbation risk notification system 456 analyzes data and identifies oncoming exacerbations based on the many different factors, such as, but not limited to, pollution, weather conditions, and other environmental factors collected in the data collection system 412. The module 456 may provide a warning to the patient or activate interventions through the interventions system 418.

The chronic risk identification module 458 identifies which issues should be addressed by patient intervention, virtual care, or escalated to the health care professional. These issues may include environmental exposure, comorbidities, lifestyle, adherence, technique, economic barriers, and SDoH barriers. The module 458 may provide a warning to the patient or activate interventions through the interventions system 418.

The data visualization module 460 takes the completed data analysis and generates interfaces for display of the data.

The refills system 416 includes a time calculation module 462 and a refill module 464. The time calculation module 462 executes an algorithm that looks at prior individual usage and predicts the time-to-refill for each individual medication for a particular patient. The algorithm may be modified to incorporate dynamic aspects such as seasonality, identified environmental sensitivity, anticipated influenza community trends, and weather forecasts to modify the predicted time-to refill. The refill module 464 determines the shipment of re-fill medication based on the predictions output by the time calculation module 462 and determines the average shipping time of the refill medication. The refill module 464 accesses medication information and usage behavior from the time calculation module 462. The refill module 464 allows coordination between an in-house healthcare provider who writes the medication and a pharmacy. Thus, the refill module 462 interfaces refill requests electronically with the supply system of the pharmacy.

The patient intervention assessment system 418 includes an adherence improvement intervention module 470, a symptom control intervention module 472, a device technique module 474, a smoking cessation module 476, a pulmonary rehabilitation module 478, a vaccine tracking module 480, a lifestyle behavior change module 482, a motivational interviewing module 484, a social support module 486, and a SDoH support module 488, in this example. Other intervention modules may be available depending on the respiratory-specific treatment steps. Other intervention modules may be managed by the system 418 that is targeted for different treatments for different ailments.

The adherence improvement intervention module 470 provides an application on the mobile device 110 directed to motivating behavior change based on focused interventions such as shared decision-making, incentives or gamification, to improve adherence by the patient. The symptom control module 472 provides the application with behavioral change focused interventions such as education, to improve symptoms experienced by a patient. The inhaler technique module 474 may be accessed if poor inhaler technique is recognized. The inhaler technique module 474 executes interfaces for technique education such as videos, access to coaching such as the coach 113, or providing surveys with technique questions. The smoking cessation module 476 is accessed if a patient reports smoking. The cessation module 476 is directed toward application for behavior change focused intervention meant to discourage continued smoking behavior. The pulmonary rehabilitation module 478 may be available if the healthcare provider recommends pulmonary rehabilitation. The module 478 provides video and coaching based pulmonary rehab exercises.

The vaccine tracking module 480 suggests vaccines for diseases (e.g., pneumonia, flu, or COVID) that may affect the patient and exacerbate conditions. The vaccine tracking module 488 may automatically book appointments for needed vaccines seasonally. The lifestyle behavior change module 482 may provide suggested interventions in relation to activities such as sleeping, eating, stress management, social support, exercise. The motivational interviewing module 484 provides connection with a coach such as the coach 113 or other health care actors. The social support module 486 provides suggestions for social support such as contacting social services, a support group or a social network. The SDoH support module 488 flags SDoH conditions and provides connection to local services such as food delivery, transportation, support groups, and assistance programs.

The intervention assessment system 418 may all provide additional data relating to the different interventions to the data collection system 412. The intervention modules of the intervention assessment system 418 result in a patient intervention protocol that may be supervised by the coach 113. The coach 113 is informed of any potential need for intervention. All contacts between the coach 113 may be collected by the data collection system 412.

The coach 113 may be in synchronous or asynchronous contact with the patient such as through telephone calls, texts, video calls, email, or communication through immersive media such as virtual reality or augmented reality. The coach 113 may also access other actors in the system such as other coaches, AI coaching health care providers and the like in relation to the patient. Multiple types of coaches/care team members possible as represented by the coach 113. Such coaching or care team members may have national health and wellness certification or health coaching certifications. They may include other actors such as ambulatory emergency care professionals, respiratory therapists or pharmacists.

Alert modules as explained above may be set up to contact the coach 113 based on patient data/analysis for syncing, adherence, exacerbation, or control transition. Technology platform to support the work—would have to be structured data. Escalation pathways may be defined if an issue is identified (e.g. Coach>care team>health care professional or Coach>Respiratory therapist>health care professional).

The medical management system 420 includes a recommendation of change module 490 and a document management module 492. Similar to the system 300, the medication management system 420 is a data analysis engine that determines a patient value associated with the treatment regimen, makes a comparison of the patient value to a threshold level, and provides a recommendation to change the step of the treatment regimen based on the comparison to the threshold level. The medical management system 420 may include a notification module that alerts the healthcare provider 112 about the system recommendation for medication management in an on-screen alert or inbox notification on a mobile device or other computer. Physician changes to the treatment regimen based on clinical recommendation from the system may be fed back into the patient action plan tracker 440 stored by the system application.

The recommendation module 490 executes a machine learning based algorithm to determine whether a change in medication should be made. The document management module 492 produces reports representing the collected data. The reports may include a 2x2 chart and associated options selected for significant features relating to the change recommendation. The document management module 492 may include data provided in a medication history format such as a timeline. Other data outputs may include graphics reflecting adherence (correct versus incorrect usage) and type of rescue (preventative versus reliever). The document management module 492 may also provide the data for interface that provides medication history and plans for a patient. Such an interface may put the current recommendation in context of the prior patient history. The recommendation engine 490 thus is a clinical decision support system that provides a holistic approach to the patient intervention. The step-up and step-down may be part of optimization of medication and other treatments for a patient. The system thus may determine which medications work best for a particular patient, and thus offer precision digital health interventions.

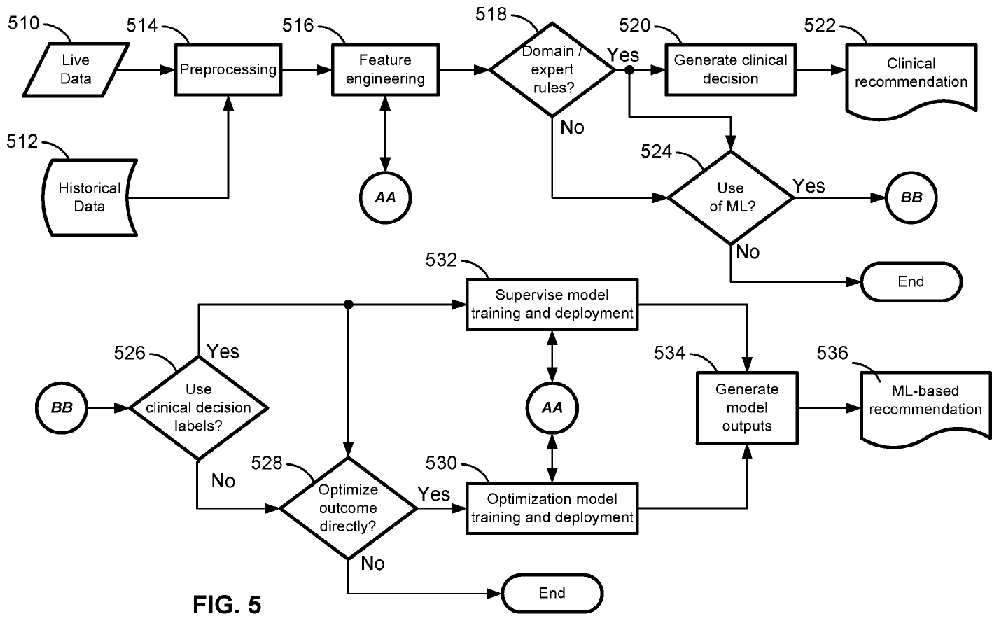

FIG. 5 is a block diagram of the recommendation machine learning process executed by medication change recommendation module 342 in FIG. 3 and the medication change recommendation module 490 in FIGS. 4A-4B. The recommendation machine learning process includes a live data input 510 and a historical data input 512. The live data may be obtained from sensors such as the sensor 120 and other devices monitoring the patient. The historical data input 512 may access historical patient data such as EHRs from databases and record keeping systems. The historical data is updated through the collection of the live data from the live data input 510.

The process in FIG. 5 encompasses two different data flows. The historical data input 512 is used for training the machine learning model while the live data input 510 is input to the trained machine learning model for a recommendation output. Thus, the live and historical data are fed into a preprocessing module 514 for the respective data flows. The training data flow includes feeding the historical data 512 into each of the modules for training the machine learning model. The historical data allows various “settings” to be learned/established (i.e. which data to use, how best to preprocess it, how is the problem being framed, which models are chosen, what are there parameters and hyperparameters). Once these have been learned, the live data 510 is fed through the modules for the data flow that generates predictions/optimizations/recommendations. In this manner, accuracy is increased by the refined settings. The efficiency of computing is also enhanced through the optimization of settings.

The preprocessing module 514 curates an exhaustive list of variables based on the input historical data to generate and append to a denormalized table. Each row of the denormalized table represents a single day of a single patient. All of the variables are assigned a column in the denormalized table. Any variables that are not available for a given patient-day are assigned a null value.

The denormalized table is fed into a feature engineering module 516. The feature engineering module 516 creates any variables explicitly required for clinical guidelines or rule-based decision system determined by a clinician or other domain expert, such as a control score, adherence level or rescue inhaler actuation count. The feature engineering module 516 determines the size of the trailing or forward window over which input or output variables will be processed. For example, the size of the trailing or forward window may be over the last 2 years, the last 30 days or the next 90 days. The feature engineering module 516 may determine the methods used for filling of null values when certain sensor data or other dynamic or static data points are unavailable. The feature engineering module 516 will also determine the aggregation methods used to summarize raw input variables up to some uniform frequency, such as an hourly or daily level, including but not limited to the mean, median, other quantiles, minimum, maximum, variance. The feature engineering module 516 will include the discretization methods of continuous variables and the one-hot encoding of categorical variables. The one hot encoding is the general process of converting a categorical variable (for example, days of the week), into a numerical format that can be interpreted by a model/algorithm. Thus, a column of weekdays may become “weekday_tuesday, weekday_wednesday, weekday_thursday, . . . ” etc. and the relevant day will be given a 1 while all other variables/columns 0.

The feature engineering module 516 includes other transformations between variables or within an individual variable's trailing window time series. This includes but is not limited to differencing, ratios and polynomial cross-terms, over any subwindow within the trailing window. For example, a new variable may be engineered that is the difference between the mean of the past 7 days of inhaler use vs. the mean of the past 30 days of inhaler use. For another example, a new variable may be engineered that is 1 when both rescue inhaler use and heart rate exceed some threshold within a day of the last week, and 0 otherwise.

A feedback loop is part of the model training process, wherein features are engineered and selected to optimize validation and test set performance during model validation. Once the optimal features are engineered and selected during the training process on a given set of historical data, they will similarly be engineered at inference time for live predictions.

After application of the feature engineering module 516, the routine determines whether domain/expert rules are to be applied for the prediction based on a selection by the end user (518). For example, Global Initiative for Asthma (GINA) guidelines may be applied. If applicable domain/expert rules are applied (518), a clinical decision may be generated based on application of the rules (520). A clinical recommendation, such as a recommendation to maintain therapy at the current levels, may then be generated (522).

The routine then decides whether to apply the machine learning model to determine a prediction for compliance (524). If the machine learning model is deployed, the routine determines whether clinical decision labels will be used (526). The clinical dependent labels are dependent variables that the machine learning model is designed to predict for the purposes of making a clinical recommendation. If the decision labels are not used (526), the routine decides whether to optimize the outcome directly (528). If the outcome is not optimized, the routine ends. If the outcome is optimized, the routine conducts optimization model training and deployment (530) as will be explained below. The optimization model training process 530 receives the outputs from the feature engineering model 516.

If the routine decides to use clinical decision labels, the routine proceeds to supervise model training and deployment for the labels (532). These labels may be based on what action a clinician actually took given a set of input conditions. In one embodiment, these labels may be broad actions such as “step-up”, “maintain” or “step-down” in relation to different tiers in a treatment regimen. In another embodiment, these labels are specific medication and dose regimens. Clinician based labels are best suited for supervised machine learning algorithms such as a generalized linear model, a tree-based model or a neural network. For example, the outputs may classify the appropriate treatment from six tiers of asthma treatments according to the GINA guidelines. The output clinical decision label may be then used as a recommendation to step-up or step-down the treatment regimen. For example, a patient at level 3 may be stepped up to level 4 based on the machine learning model output label. The output clinical recommendation is an objective output that removes the subjectivity from human healthcare providers using individual judgement and/or incomplete information to make treatment step-up or step-down recommendations.

The labels may also be based on the change over some forward window in a patient condition or a novel combination of patient conditions that is a proxy for disease control, including but not limited to a control score (ACT/CAT), a count of daily SABA puffs or other relevant biomarkers. Other patient condition labels are best suited for certain optimization algorithms that attempt to directly maximize or minimize a dependent variable within certain constraints such as model predictive control or clustering.

The routine thus will conduct supervised model training and deployment for an output of specified clinician decision labels (532). The system will train and validate a model or ensemble of models on the historical inputs and outputs. The training process 532 receives the outputs from the feature engineering model 516. These models are designed to predict what the most likely action a clinician would take given a current set of inputs. In one embodiment, a single model is trained on a single output vector representing broad actions such as “step-up”, “maintain” or “step-down”. In another embodiment, a single model is trained on multi-output labels involving specific medication and dose regimens. In yet another embodiment, multiple models are trained on different subsets of inputs and outputs in order to produce an ensemble of models or multiple models in sequence to produce a probability distribution over medications and doses.

Any training process involves best practice for longitudinal cross validation, and may include a resampling of the historical data in order to more accurately reproduce the distribution of disease states, other relevant inputs and medication regimens across a given geography or relevant patient population. Such a resampling may involve a novel combination with data sets including but not limited to industry sales data and government health data in order to achieve a representative training distribution of the population of interest. During cross-validation, patient data is partitioned either by time, patient or both time and patient. Any outer validation set for a given training run should exist in the future from the training set.

Training the model is thus a process in which the model tries to predict the label for each data point, and the parameters of the model are tuned based on the difference between the prediction and the label. The function used to calculate this difference is called the loss function. The loss function determines how to change each model parameter based on the model type and the prediction-label difference.

In this example, the classification problem where the goal is to predict step-up or step-down may use cross-entropy loss. In a regression problem where numerical change in recommended dosage is predicted, mean-squared-error loss may be used. Different loss functions for a single type of problem may be run and analyzed, since the choice of loss function affects training behavior and therefore performance.

Using the machine learning model in these use cases is beneficial because the model may be continuously trained on the data received from patient by the system 400. This allows the model to adjust decision-making parameters based on live data every day, which would be difficult to do manually or with a rule-based model, and also enables lifelong learning without requiring complete retraining of the model. Using machine learning also allows analysis of with different model types, since different interpretable ML models may give different insight into the collected data.

Once the models are trained and deployed, the data flow for the live data input 510 may be generated for obtaining recommendation outputs. The live data is fed to the preprocessing module 514. The pre-processed data is then fed to the feature engineering module 516 and the recommendation process is initiated. The routine allows the models to generate outputs (534). The final outputs may include, but are not limited to, rule-based decision from clinical guideline, probabilities or representations of probability distributions based on the most likely clinical decision given a set of inputs, or a direct data-driven recommendation based solely on the output of an optimization algorithm, or any combination thereof. Further outputs may include any numerical or visual representation that aids end users in understanding, interpreting and validating the recommendation or recommendations. The outputs are then formed into recommendations for changes, if applicable, to a treatment regimen (536).

Supervised algorithms include but are not limited to generalized linear models, tree-based models or neural networks. In one embodiment, a tree-based model produces a novel, highly interpretable decision tree that clinicians can understand and trust, while yielding deeper understanding of the relationships between existing and novel variables and their relationship to disease management decisions that are not captured by current decision making tools. In another embodiment, neural networks with architectures particularly well suited to this class of supervised time series prediction problem, such as a time convolutional network, are used to maximize the fidelity of input data while also being well-suited to multi-output labels. For example, rather than feature engineering summary statistics such as the mean and variance of a preprocessed variable over a trailing window to create a lossy input vector, each individual daily data point can be passed in as an input matrix. In another model embodiment, a stochastic model may be used, such as a hidden Markov model, where the hidden states represent some latent representation of disease control. Such a representation may also be further valuable to clinicians by revealing new relationships between input variables and disease control.

In selecting an optimization model 530, clinical decision labels may be avoided. The decision labels are avoided when a desired measure of disease control is instead used directly as the label or dependent variable, and the problem is treated as an optimization problem to maximize disease control over some finite forward window. In one embodiment, the optimization problem may be solved by clustering historical data and selecting the clinical decision that yields the largest improvement in some measure of disease control over a forward window such as a CAT or ACT control score. Such an approach may yield novel disease phenotypes based on a broad array of biomarkers and other variables, while also allowing clinicians to observe what treatment has been most effective in the past given similar input conditions. In another embodiment, the problem is treated as a model predictive control problem, where again the dependent variable(s) are some measure(s) of disease control, the independent variables that can be controlled such as the choice of medication and dose, and independent variables that cannot be controlled are treated as disturbances. Such a model then seeks to optimize the dependent variable within any constraints (for example, the financial or health-related costs of a certain medication).

Once the medications are prescribed based on the step of the treatment regimen, the system 100 may continue to monitor the use of such medication via the application executed on the patient computing device 110 in FIG. 1 and collect the data described above. The system 100 may also provide reminders to patients based on a medication treatment plan, i.e., to encourage adherence. The system 100 may also provide helpful information such as providing a user interface that includes visual or written directions to entering an injection location and walks them through the steps of injection.

Such an application may include a dashboard interface that allows a patient to enter occurrences of taking the medication as part of the enhanced treatment. The interface may also collect patient reported outcomes from the administration of the medication. This data may be reported to the application server 130 along with prescription and dispensing information from the pharmaceutical supply server 180 to track the medications for a particular patient based on the system 300 in FIG. 3. The application may also include interfaces with educational information on respiratory ailments and the treatments such as the particular medication.

An application for execution by a computer device available to the healthcare provider 112 may include trend graphics showing the historic record of previous medication and treatment that may be made available from the systems 300 and 400. Such a historic record may be interposed with other patient data such as ACT history, adherence, or inhaler actuation events.

The system 400 in FIGS. 4A-4B may be used to streamline re-ordering of medications and provide reminders to re-order the medications. The system 400 can track shipping status to increase medication fulfillment and adherence. The use data as well as other patient data may be transmitted to a pharmaceutical distribution system that may include the pharmaceutical supply server 180 in FIG. 1. The tracking of the patient and the use of the medication allows notifications to be sent to the patient, caregiver, healthcare provider, or other parties to predict re-ordering of the biologics.

As explained above, the application may include an interface for self-reporting by the patient. Such data may include self-reported ACT data, self-reported quality of life, and self-reported exacerbations such as hospitalization, prescriptions of oral corticosteroids (OCS), or appointments with healthcare providers. Other sensor data may be collected by the system 100 that may be included in the analysis of the responsiveness to a specific medication. These may include passive activity, sleep data, at-home spirometry readings, and response to pollutants and irritants. Thus, night time awakenings may be characterized by night time inhaler use, or a sleep tracking sensor. For example, the system could be linked to sleep data from a wearable sensor, such as a FitBit, or a phone application such as the application available from Sleep Score Labs. Another source of data may include a data connection to a smart-syringe for biologics. Such smart syringes are known in the art, and could be connected to the current system to confirm use of the biologic, time and date of use, quantity of drug, batch of drug, etc.

FIG. 6A shows a screen image of an example patient summary interface 600 generated for a computing device such as the computing device 110 operated by the healthcare professional 112 in FIG. 1. The computing device 110 includes an application that accesses either the system 300 in FIG. 3 or the system 400 in FIG. 4A-4B for step-up or step-down recommendations in the treatment regimen. The interface 600 includes a dashboard option that summarizes all patients associated with the healthcare provider and a patients option that allows data on specific patients to be displayed. In this example, the interface 600 is when the dashboard option is selected.

The interface 600 includes a patient summary area 610 that provides a summary of patients associated with the healthcare provider. The summary area 610 includes a high-risk field 612, an increasing risk field 614, a consultation request field 616, a step-up field 618 and a step-down field 620. Selecting any of the fields 612, 614, 616, 618 or 620 allows a listing of patients falling under the category associated with the field in a patient data area 622. Thus, a healthcare provider may select one of the fields and all patients associated with the condition will be displayed to allow the healthcare provider to take action in relation to those patients if required. An environment field 624 displays helpful environmental data for the geographical area associated with the healthcare provider such as the temperature, air quality, humidity, and wind speed.

The high-risk field 612 shows the number of patients associated with the healthcare provider that have been identified from the system 300 or system 400 as having a high exacerbation risk. The increasing risk field 614 shows the number of patients associated with the healthcare provider that have been identified from the system 300 or system 400 as having an increasing risk of exacerbation. The consultation request field 616 shows the number of patients associated with the healthcare provider that have requested a consultation. The step-up and step-down fields 618 and 620 shows the number of patients associated with the healthcare provider that have been identified from the system 300 or system 400 as recommended for a step-up or a step-down in treatment.

In this example, the step-up field 618 has been selected. The step-up field 618 shows two patients for which a step-up in treatment has been recommended. Thus, two patient records 630 are shown in the patient data area 622. Each patient record 630 includes a patient information field 632, an at-a-glance field 634, a details field 636, and a recommendation field 638. The patient information field 632 includes the name of the patient, the age of the patient and a picture of the patient. The at-a-glance field 634 includes various icons that represent increases or decreases in rescue use and adherence. Thus, an inhaler icon shows a percentage increase in rescue use. The details field 636 shows the different factors that influence the recommendation for the step-up treatment. In this example, the details field shows the adherence percentage, an increase in rescue use that supports a step-up in treatment, and visits to the emergency room. The recommendation field 638 includes a details button that allows the display of further information when selected.

FIG. 6B is a screen image of a patient data interface 640 generated for a computing device such as the computing device 110 operated by the healthcare professional 112 in FIG. 1 when the details button in the recommendations field 638 in FIG. 6A is selected. The computing device 110 includes an application that accesses either the system 300 in FIG. 3 or the system 400 in FIG. 4A-4B for step-up or step-down recommendations specific to the patient on the interface 640. The interface 640 includes a patient data field 642 and a recommendation graph 644. The patient data field 642 includes the name of the patient, date of birth, special notes, any comorbidities, and the type of ailment such as COPD or asthma. The interface 640 includes a record outreach button 646 and a contact button 648 to enable and track clinician action. The record outreach button 646 allows the user to write a note that a staff member reached out to a patient. The contact button 648 allows the user to directly reach out to a patient through the platform via media such as a message, a telehealth call, or a video conference. Other useful graphics may be shown in the interface 640 such as the history of adherence or rescue usage or other key signals, such as activity levels.

The recommendation graph 644 plots a recommendation based on relevant factors. In this example, the graph 644 shows a vertical scale of rescue usage and a horizontal scale of adherence. The vertical and horizontal scales define four quadrants representing an action. Depending on the data, the graph 644 may recommend a step-up, a step-down, a stay the course, or providing more education in inhaler technique or other information relevant to the ailment. Each of these are provided in one of the four quadrants of the graph 644. In this example, the patient has high rescue usage and high adherence and thus a quadrant representing step-up is highlighted. The highlighted step-up quadrant includes a link 650 to provide further details of the step-up in treatment. The thresholds of the graph 644 may be adjusted by selecting a threshold link 652.

FIG. 6C shows a screen image of a recommendation details display 660 that is displayed when the details link 650 is selected in FIG. 6B. The recommendation details display 660 includes information that supports the step-up recommendation as determined by the machine learning model. A similar display may be shown for information supporting a step-down recommendation. In this example, there are four factors that support the step-up recommendation. The number of factors and types of factors shown may be adjusted. All of the displayed factors are over thresholds that may be further selected by the healthcare provider for the patient for a step-up in treatment. In this example, the display 600 includes an adherence field 662, a rescue medication usage field 664, a test results field 666, and a visits field 668. The adherence field 662 includes a description of the adherence percentage over 30 days for the patient and a graph that shows the adherence percentage in a solid bar relative to the set threshold as a dashed line. The rescue medication usage field 664 includes a description of the level of rescue medication as well as a graph showing the usage (in puffs per day) in a solid bar relative to the usage threshold that is shown as a dashed line. The test results field 666 shows the score of the most recent ACT taken by the patient as a bar relative to the threshold score. The visits field 668 shows a description of the number of hospital or ER visits made by the patient.

FIG. 6D shows a screen image of a customization panel display 670 that allows the healthcare provider to customize the step-up and step-down threshold factors overall, effectively over-riding the machine learning generated recommendations. The notification display includes a step-up threshold selection area 672 and a step-down threshold selection area 674. The step-up threshold selection area 672 includes a toggle switch 680 allowing a healthcare provider to select which factors they want to consider for a step-up recommendation and at what threshold. The toggle switches include a selection switch 682 for setting the percentage of control medication adherence over a selected period of time. Another toggle switch 684 allows setting the rescue medication usage level over a selected period of time. Another toggle switch 686 allows setting the threshold number of ER or clinic visits over a selected period of time. Another toggle switch 686 allows setting the threshold score for an ACT for the patient to justify a step-up in treatment. Thus, in this example since the toggle switches 682, 684, and 686 are selected, the healthcare provider will receive a step-up recommendation when the control adherence exceeds 75% over 1 month, when the rescue medication usage is over 6 puffs per day over 1 month, and if there is more than 1 visit to an ER or clinic in one month.

Similarly, the step-down threshold selection area 674 includes a toggle switch 690 allowing a healthcare provider to select notification for a step-down recommendation. The selection area 674 includes toggle switches for setting different thresholds and whether a notification will occur if the condition is met. The toggle switches include a selection switch 692 for setting the percentage of control medication adherence over a selected period of time. Another toggle switch 694 allows setting the rescue medication usage level over a selected period of time. Another toggle switch 696 allows setting the threshold number of ER or clinic visits over a selected period of time. Another toggle switch 696 allows setting the threshold score for an ACT for the patient to justify a step-down in treatment. Thus, in this example since the toggle switches 692, 694, and 696 are selected, the healthcare provider will receive notifications when the control adherence falls under 50% over 1 month, when the rescue medication usage is under 5 puffs per day over 1 month, or if there is an ACT score under 15.

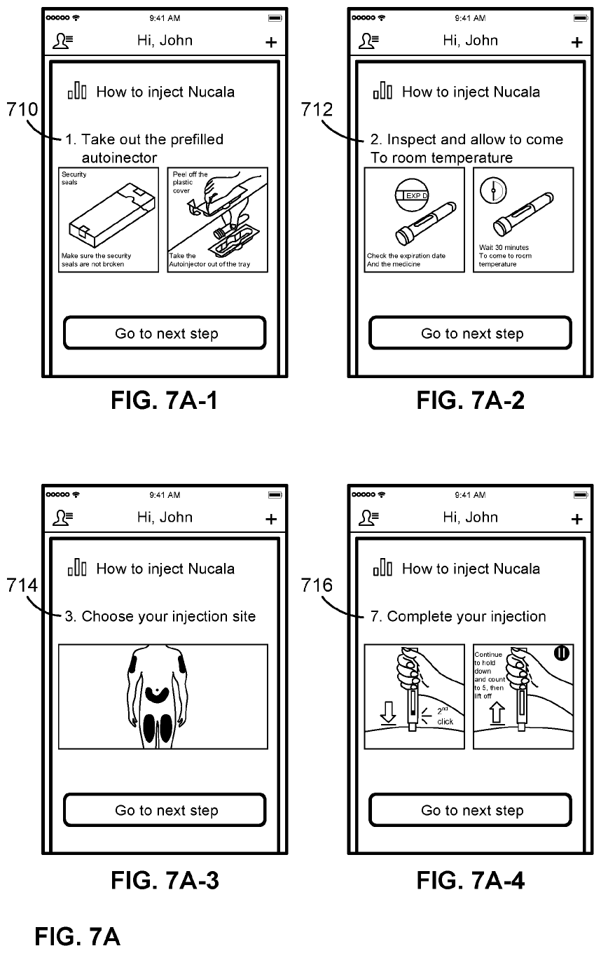

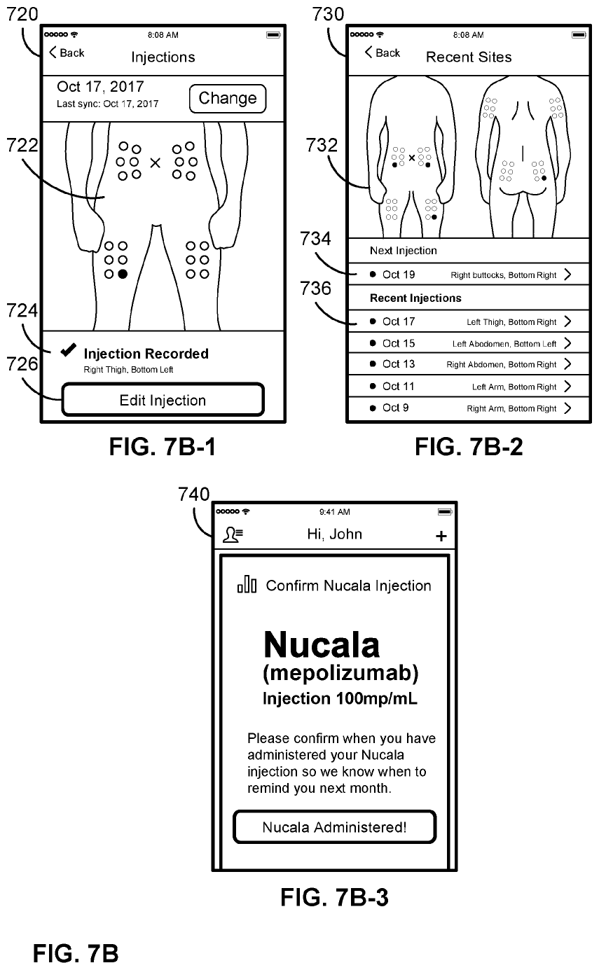

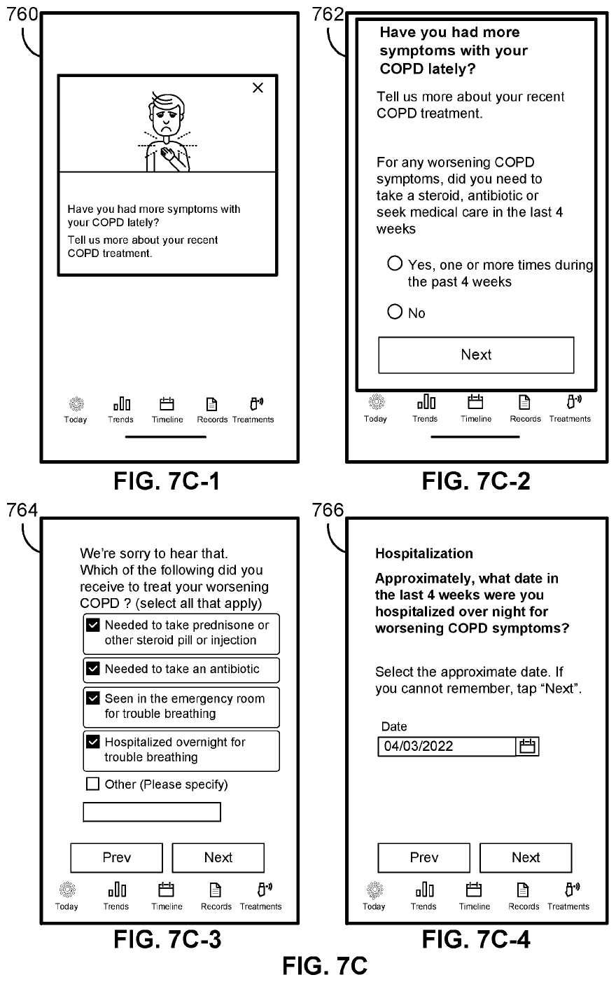

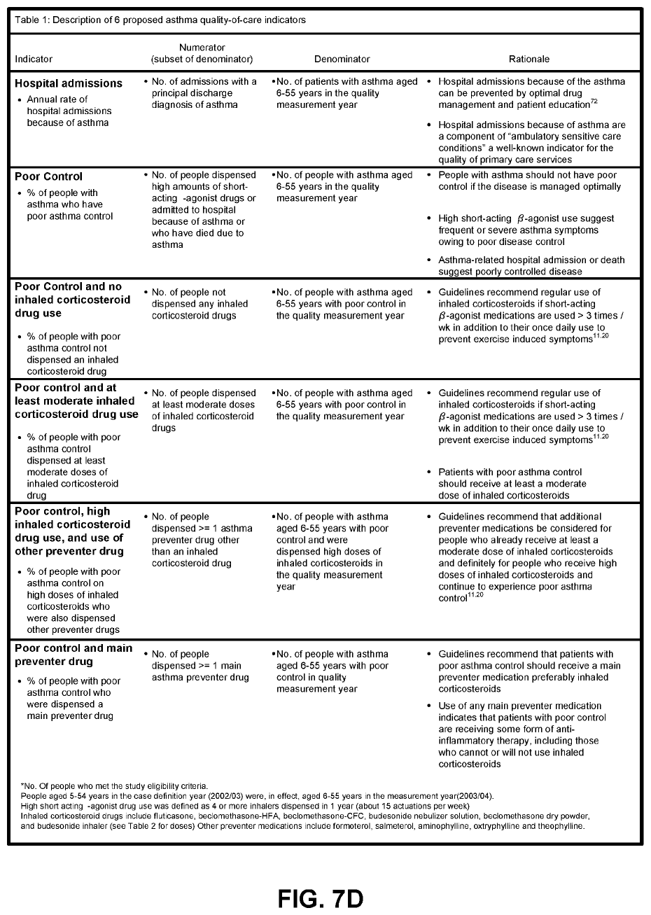

FIG. 7A is a series of screen images of interfaces for instructing a patient on making an injection of medication that may be shown on a user device. A first interface 710 shows graphics instructing the patient to remove the injector from the package. The user may select a next step button that will lead to the display of an interface 712 that shows graphics instructing a user to wait for the injector to come to room temperature. After completion of the wait, the user may select a next step button that will display an interface 714 that instructs a user to choose an injection site from a graphic of different body locations for the injection. Once the user chooses a location, other graphics are shown such as the interface 716 that instruct the user on use of the injector on the selected site. The application on the user device may report the display of the instruction interfaces in FIG. 7A to the medication tracking module 410 in FIGS. 4A-4B.

FIG. 7B is a series of screen images allowing a patient to record the injection of medication such as a biologic. An injections interface 720 includes an image of different injection locations 722. An injection description 724 is displayed that show where injections have been recorded. An edit button 726 allows the patient to edit the location of the injection and other data relating to the injection. Another interface 730 shows an image of different injection locations 732. A next injection field 734 shows the scheduled next injection and the location of the next injection. A historical field 736 shows the past injections and the locations of the past injections. A record interface 740 allows a patient to confirm the injection of the medication. The data indicating the injection, location of the injection, and time of the injection may be collected by the active collection module 424 in FIGS. 4A-4B.

FIG. 7C is a series of screen images displayed on a user device allowing a patient to record information for determining potential exacerbations that may be collected by exacerbation tracker 442 in FIGS. 4A-4B. A first interface 760 is an introduction question that may be triggered by signs of exacerbations. A second interface 762 includes a question for whether medication was necessary. A third interface 764 requests information of treatments for the exacerbations through check off selections. A final interface 766 requests information relating to hospitalizations due to the exacerbations. The collected data from the interfaces 762, 764, and 766 may be sent from the user device to the exacerbation tracker 442.

FIG. 7D is a table that shows example indicators to evaluate if quality of care is satisfied before a decision of step-up/step-down treatment for a respiratory ailment such as asthma is made. EMR data and/or claims data from the modules 432 or 434 may be collected to evaluate these indicators.

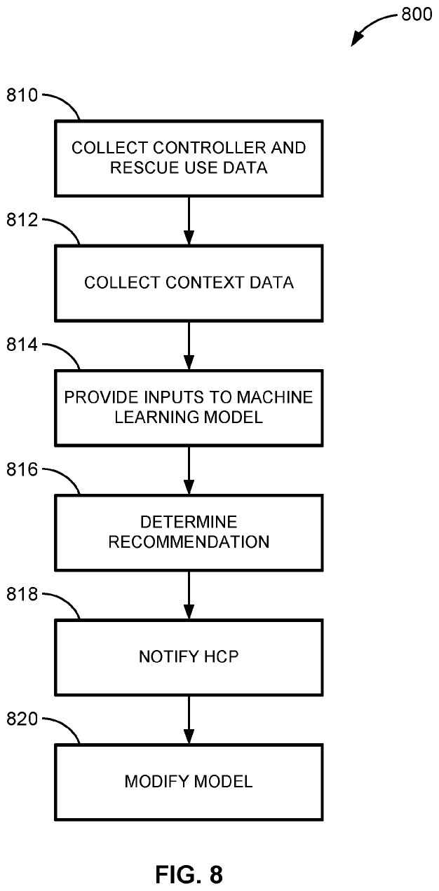

FIG. 8 is a flow diagram 800 of a data collection and analysis routine to collect data and provide step-up or step-down recommendations according to certain aspects of the present disclosure. The flow diagram in FIG. 8 is representative of example machine readable instructions for the process of collecting data and selecting patients for enhanced treatment. In this example, the machine readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithm is described with reference to the flowchart illustrated in FIG. 8, persons of ordinary skill in the art will readily appreciate that many other methods of implementing the example machine readable instructions may alternatively be used. For example, the order of execution of the blocks may be changed, and/or some of the blocks described may be changed, eliminated, or combined.

The routine first collects controller and rescue use data from a general population of patients that use respiratory medications (810). The use data is collected over a period of time such as 90 days. The routine collects other contextual data such as patient demographics and environmental data from external sources (812). The routine then provides the collected data to the trained machine learning model for outputs of step-up or step-down recommendations (814).

The routine then determines patients which meet the clinical labels for step-up or step-down recommendations (816). The routine then provides a notification for the associated healthcare providers for all patients with a step-up or step-down recommendation (818). The routine stores the collected data and modifies the machine learning model (820).

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

One or more elements or aspects or steps, or any portion(s) thereof, from one or more of any of claims 1-41 below can be combined with one or more elements or aspects or steps, or any portion(s) thereof, from one or more of any of the other claims 1-41 or combinations thereof, to form one or more additional implementations and/or claims of the present disclosure.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. A method of determining a recommendation to change a treatment regimen of a respiratory ailment, the treatment regimen including a plurality of steps, the method comprising:
collecting use data of a respiration medicament device to deliver controller or rescue respiration medicament to the patient via a communication interface;
transmitting the use data to a storage device;
storing the use data in the storage device accessible to a data analysis engine; based on the collected use data and patient context data, determining a patient value associated with the treatment regimen via the data analysis engine;
making a comparison of the patient value to a threshold level;
providing a recommendation to change the step of the treatment regimen based on the comparison to the threshold level; and
providing a notification of recommendation of the change of the step of the treatment regimen.

2. The method of claim 1, wherein the respiratory ailment is asthma or COPD.

3. The method of claim 1, wherein each step of the treatment regimen includes an associated controller respiration medicament or a rescue respiration medicament.

4. The method of claim 1, wherein the treatment regimen is defined by Global Initiative for Asthma (GINA) guidelines or the National Heart Lung Blood Institute guidelines.

5. The method of claim 1, wherein the threshold level is one of an adherence percentage over a period of time, a number of rescue medicaments over a period of time, a score on a respiratory test, or a visit to a treatment facility.

6. The method of claim 5, wherein the respiratory test is one of the COPD assessment test (CAT) or the asthma control test (ACT).

7. The method of claim 1, wherein the recommendation is a step-up in treatment when the comparison is above the threshold level.

8. The method of claim 1, wherein the recommendation is a step-down in treatment when the comparison is below the threshold level.

9. The method of claim 1, wherein the patient context data is one of demographic data, environmental data, weather data, and social determinants of health.

10. The method of claim 9, wherein the demographic data is collected from interfacing with an electronic health record system or self-reported by the patient.

11. The method of claim 1, wherein the recommendation of the change is made at a predetermined interval over a period of time.

12. The method of claim 1, further comprising inputting the collected data to a machine learning model to output the threshold value, wherein the machine learning model is trained from collected context data, use data, and outcomes from the treatment regimen from a population of patients.

13. The method of claim 12, wherein the machine learning model includes a clinical decision label for the threshold output.

14. The method of claim 12, wherein the machine learning model is based on one of a generalized linear model, a tree-based model or a neural network.

15. The method of claim 1, further comprising categorizing the use between preemptive or emergency use.

16. The method of claim 1, further comprising outputting a report of the collected use and context data.

17. The method of claim 1, further comprising collecting the context data through a survey displayed by an application executed by a mobile computing device operated by the patient.

18. The method of claim 1, wherein the notification is provided electronically to one of the patient, a caregiver, or a healthcare provider.

19. The method of claim 1, further comprising ordering additional medication for the patient through a medication supply system based on the recommendation.

20.-21. (canceled)

22. A system to determine changing a treatment regimen having a plurality of treatment steps, the system comprising:
a communication interface to collect use data of a respiration medicament device to deliver controller or rescue respiration medicament to a patient;
a storage device to store the collected use data; and
a data analysis engine configured to:
input the use data and context data of patient to determine a patient value; comparing the patient value with a threshold value;
based on the comparison, making a recommendation to change the treatment regimen; and
providing a notification of the recommendation.

23. The system of claim 22, wherein the respiratory ailment is asthma or COPD.

24. The system of claim 22, wherein each step of the treatment regimen includes an associated controller respiration medicament or a rescue respiration medicament.

25. The system of claim 22, wherein the treatment regimen is defined by Global Initiative for Asthma (GINA) guidelines or the National Heart Lung Blood Institute guidelines.

26. The system of claim 22, wherein the threshold level is one of an adherence percentage over a period of time, a number of rescue medicaments over a period of time, a score on a respiratory test, or a visit to a treatment facility.

27. The system of claim 26, wherein the respiratory test is one of the COPD assessment test (CAT) or the asthma control test (ACT).

28. The system of claim 22, wherein the recommendation is a step-up in treatment when the comparison is above the threshold level.

29. The system of claim 22, wherein the recommendation is a step-down in treatment when the comparison is below the threshold level.

30. The system of claim 22, wherein the patient context data is one of demographic data, environmental data, weather data, and social determinants of health.

31. The system of claim 30, wherein the demographic data is collected from interfacing with an electronic health record system or self-reported by the patient.

32. The system of claim 22, wherein the recommendation of the change is made at a predetermined interval over a period of time.

33. The system of claim 22, wherein the data analysis engine determines the threshold value from inputting the collected data to a machine learning model to output the threshold value, wherein the machine learning model is trained from collected context data, use data, and outcomes from the treatment regimen from a population of patients.

34. The system of claim 33, wherein the machine learning model includes a clinical decision label for the recommendation output.

35. The system of claim 33, wherein the machine learning model is based on one of a generalized linear model, a tree-based model or a neural network.

36. The system of claim 22, wherein the data analysis engine is further configured to categorize the use between preemptive or emergency use.

37. The system of claim 22, wherein the data analysis engine is further configured to output a report of the collected use and context data.

38. The system of claim 22, wherein the context data is collected through a survey displayed by an application executed by a mobile computing device operated by the patient.

39. The system of claim 22, wherein the notification is provided electronically to one of the patient, a caregiver, or a healthcare provider.

40. The system of claim 22, wherein the data analysis engine is further configured to order additional medication for the patient through a medication supply system based on the recommendation.

41. A method of training a machine learning model to determine a threshold for recommendation of one step of a multiple step treatment regimen for a respiratory ailment, the method comprising:
collecting use data from the use of a respiratory medication application device from each of a population of patients undergoing one of the steps of the multiple step treatment regimen;
collecting contextual data from each of the population of patients;
collecting outcomes from the population of patients for the treatment regimen; determining initial recommendation thresholds for each of the steps of the multiple step treatment regimen; and
adjusting the initial recommendation thresholds based on the collected use data, contextual data, and outcomes until a predetermined level of confidence is reached.

42. A non-transient computer-readable storage medium having instructions embodied thereon, the instructions being executable by one or more processors to perform a method determining a recommendation to change a treatment regimen of a respiratory ailment, the treatment regimen including a plurality of steps, the method comprising:
collecting use data of a respiration medicament device to deliver controller or rescue respiration medicament to the patient via a communication interface;
transmitting the use data to a storage device;
storing the use data in the storage device accessible to a data analysis engine; based on the collected use data and patient context data, determining a patient value associated with the treatment regimen via the data analysis engine;
making a comparison of the patient value to a threshold level;
providing a recommendation to change the step of the treatment regimen based on the comparison to the threshold level; and
providing a notification of recommendation of the change of the step of the treatment regimen.

43. The non-transient computer-readable storage medium of claim 42, wherein the respiratory ailment is asthma or COPD.

44. The non-transient computer-readable storage medium of claim 42, wherein each step of the treatment regimen includes an associated controller respiration medicament or a rescue respiration medicament.

45. The non-transient computer-readable storage medium of claim 42, wherein the treatment regimen is defined by Global Initiative for Asthma (GINA) guidelines or the National Heart Lung Blood Institute guidelines.

46. The non-transient computer-readable storage medium of claim 42, wherein the threshold level is one of an adherence percentage over a period of time, a number of rescue medicaments over a period of time, a score on a respiratory test, or a visit to a treatment facility.

47. The non-transient computer-readable storage medium of claim 46, wherein the respiratory test is one of the COPD assessment test (CAT) or the asthma control test (ACT).

48. The non-transient computer-readable storage medium of claim 42, wherein the recommendation is a step-up in treatment when the comparison is above the threshold level.

49. The non-transient computer-readable storage medium of claim 42, wherein the recommendation is a step-down in treatment when the comparison is below the threshold level.

50. The non-transient computer-readable storage medium of claim 42, wherein the patient context data is one of demographic data, environmental data, weather data, and social determinants of health.

51. The non-transient computer-readable storage medium of claim 50, wherein the demographic data is collected from interfacing with an electronic health record system or self-reported by the patient.

52. The non-transient computer-readable storage medium of claim 42, wherein the recommendation of the change is made at a predetermined interval over a period of time.

53. The non-transient computer-readable storage medium of claim 42, further comprising inputting the collected data to a machine learning model to output the threshold value, wherein the machine learning model is trained from collected context data, use data, and outcomes from the treatment regimen from a population of patients.

54. The non-transient computer-readable storage medium of claim 53, wherein the machine learning model includes a clinical decision label for the threshold output.

55. The non-transient computer-readable storage medium of claim 53, wherein the machine learning model is based on one of a generalized linear model, a tree-based model or a neural network.

56. The non-transient computer-readable storage medium of claim 42, further comprising categorizing the use between preemptive or emergency use.

57. The non-transient computer-readable storage medium of claim 42, further comprising outputting a report of the collected use and context data.

58. The non-transient computer-readable storage medium of claim 42, further comprising collecting the context data through a survey displayed by an application executed by a mobile computing device operated by the patient.

59. The non-transient computer-readable storage medium of claim 42, wherein the notification is provided electronically to one of the patient, a caregiver, or a healthcare provider.

60. The non-transient computer-readable storage medium of claim 42, further comprising ordering additional medication for the patient through a medication supply system based on the recommendation.
